(19) United States
(12) Patent Application Publication
(10) Pub. No.: US 2022/0031772 A1
(43) Pub. Date: Feb. 3, 2022

(54) COMPOSITIONS COMPRISING BACTERIAL STRAINS

(71) Applicant: 4D Pharma Research Limited, Aberdeen (GB)

(72) Inventors: Samantha YUILLE, Aberdeen (GB); Helene SAVIGNAC, Aberdeen (GB); Sarah REID, Aberdeen (GB); Imke Elisabeth MULDER, Aberdeen (GB); Nicole REICHARDT, Aberdeen (GB); Emma RAFTIS, Aberdeen (GB)

(21) Appl. No.: 17/231,247

(22) Filed: Apr. 15, 2021

Related U.S. Application Data
(63) Continuation of application No. PCT/EP2019/078598, filed on Oct. 21, 2019.

(30) Foreign Application Priority Data
Oct. 19, 2018 (EP) 18201603.0

(51) Int. Cl. A61K 35/742; A61P 37/06

(52) U.S. Cl. CPC A61K 35/742; C12R 2001/145; A61P 37/06

(57) ABSTRACT

The invention provides compositions comprising a bacterial strain of the species Blautia producta or Blautia coccoides, for use in therapy.

### CROSS-REFERENCE

This application is a continuation of International Application No. PCT/EP2019/078598, filed Oct. 21, 2019, which claims the benefit of European Application No. 18201603.0, filed Oct. 19, 2018, all of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Apr. 12, 2021, is named 56708_750_301_SL.txt and is 5,950 bytes in size.

### TECHNICAL FIELD

This invention is in the field of compositions comprising bacterial strains isolated from the mammalian digestive tract and the use of such compositions in the treatment of disease.

### BACKGROUND TO THE INVENTION

The human intestine is thought to be sterile in utero, but it is exposed to a large variety of maternal and environmental microbes immediately after birth. Thereafter, a dynamic period of microbial colonization and succession occurs, which is influenced by factors such as delivery mode, environment, diet and host genotype, all of which impact upon the composition of the gut microbiota, particularly during early life. Subsequently, the microbiota stabilizes and becomes adult-like [1]. The human gut microbiota contains more than 500-1000 different phylotypes belonging essentially to two major bacterial divisions, the Bacteroidetes and the Firmicutes [2]. The successful symbiotic relationships arising from bacterial colonization of the human gut have yielded a wide variety of metabolic, structural, protective and other beneficial functions. The enhanced metabolic activities of the colonized gut ensure that otherwise indigestible dietary components are degraded with release of by-products providing an important nutrient source for the host. Similarly, the immunological importance of the gut microbiota is well-recognized and is exemplified in germfree animals which have an impaired immune system that is functionally reconstituted following the introduction of commensal bacteria [3-5].

Dramatic changes in microbiota composition have been documented in gastrointestinal disorders such as inflammatory bowel disease (IBD). For example, the levels of Clostridium cluster XIVa bacteria are reduced in IBD patients whilst numbers of E. coli are increased, suggesting a shift in the balance of symbionts and pathobionts within the gut [6-9]. Interestingly, this microbial dysbiosis is also associated with imbalances in T effector cell populations.

In recognition of the potential positive effect that certain bacterial strains may have on the animal gut, various strains have been proposed for use in the treatment of various diseases (see, for example, [10-13]). Also, certain strains, including mostly Lactobacillus and Bifidobacterium strains, have been proposed for use in treating various inflammatory and autoimmune diseases that are not directly linked to the intestines (see [14] and [15] for reviews). Consortium compositions comprising many bacterial strains have been proposed for use in treating diseases such as graft-versus-host disease (see [16]), but the identity of the strains used and the species that they belong to was not definitively determined. In summary, the relationship between different diseases and different bacterial strains, and the precise effects of particular bacterial strains on the gut and at a systemic level and on any particular types of diseases, are poorly characterised.

There is a requirement in the art for new methods of treating diseases. There is also a requirement for the potential effects of gut bacteria to be characterised so that new therapies using gut bacteria can be developed.

### SUMMARY OF THE INVENTION

The inventors have developed new compositions comprising bacterial strains of the species Blautia producta and Blautia coccoides that can be used for treating and preventing inflammatory and autoimmune diseases. In particular embodiments, the invention provides a composition comprising a bacterial strain of the species Blautia producta or Blautia coccoides, for use in a method of treating or preventing a disease or condition selected from the group consisting of: graft-versus-host disease (GVHD); inflammatory bowel diseases, such as Crohn's disease or ulcerative colitis; asthma, such as allergic asthma or neutrophilic asthma; arthritis, such as rheumatoid arthritis, osteoarthritis, psoriatic arthritis, or juvenile idiopathic arthritis; multiple sclerosis; psoriasis; systemic lupus erythematosus; and allograft rejection.

The inventors have identified that treatment with Blautia producta strains reduces gut permeability in a mouse model of disease. Increased gut permeability is associated with many inflammatory and autoimmune diseases. Blautia coccoides is extremely similar to Blautia producta. Thus, the compositions of the invention may be useful in the treatment of inflammatory or autoimmune diseases.

In some embodiments, bacterial strains from the species Blautia producta or Blautia coccoides may provide therapeutic benefits in the treatment or prevention of GVHD. The inventors have identified that treatment with Blautia producta strains increase survival from GVHD in a mouse model of disease. Blautia coccoides is extremely similar to Blautia producta. The strains of the invention may therefore be useful in the treatment or prevention of GVHD. In certain embodiments, the compositions of the invention are for use in the treatment or prevention of GVHD in a subject. In preferred embodiments, the invention provides a composition comprising a bacterial strain of the species Blautia producta or Blautia coccoides for use in the treatment or prevention of GVHD.

In some embodiments the invention provides a composition comprising a bacterial strain of the species Blautia producta or Blautia coccoides for use in a method of treating or preventing an inflammatory bowel disease. The inventors have identified that treatment with Blautia producta strains reduces severity of colitis in a mouse model of disease. Blautia coccoides is extremely similar to Blautia producta. Thus, the compositions of the invention may be useful in the treatment of inflammatory diseases. In some embodiments, the compositions of the invention are for use in the treatment or prevention of inflammatory bowel disease. In some embodiments, the compositions of the invention are for use in the treatment or prevention of ulcerative colitis. In some embodiments, the compositions of the invention are for use in the treatment or prevention of Crohn's disease. In certain embodiments, the compositions of the invention are for use in reducing ulcerations and/or bleeding in the treatment of an inflammatory bowel disease, in particular in the treatment of colitis and ulcerative colitis.

In some embodiments, bacterial strains from the species Blautia producta or Blautia coccoides may provide therapeutic benefits in the treatment or prevention of asthma, such as allergic asthma or neutrophilic asthma. In certain embodiments, the compositions of the invention are for use in the treatment or prevention of asthma in a subject. In certain embodiments, the invention provides a composition comprising a bacterial strain of the species Blautia producta or Blautia coccoides for use in the treatment or prevention of asthma.

In some embodiments, bacterial strains from the species Blautia producta or Blautia coccoides may provide therapeutic benefits in the treatment or prevention of arthritis, such as rheumatoid arthritis, osteoarthritis, psoriatic arthritis, or juvenile idiopathic arthritis. In certain embodiments, the compositions of the invention are for use in the treatment or prevention of arthritis in a subject. In certain embodiments, the invention provides a composition comprising a bacterial strain of the species Blautia producta or Blautia coccoides for use in the treatment or prevention of arthritis.

In some embodiments, bacterial strains from the species Blautia producta or Blautia coccoides may provide therapeutic benefits in the treatment or prevention of multiple sclerosis. In certain embodiments, the compositions of the invention are for use in the treatment or prevention of multiple sclerosis in a subject. In certain embodiments, the invention provides a composition comprising a bacterial strain of the species Blautia producta or Blautia coccoides for use in the treatment or prevention of multiple sclerosis.

In some embodiments, bacterial strains from the species Blautia producta or Blautia coccoides may provide therapeutic benefits in the treatment or prevention of psoriasis. In certain embodiments, the compositions of the invention are for use in the treatment or prevention of psoriasis in a subject. In certain embodiments, the invention provides a composition comprising a bacterial strain of the species Blautia producta for Blautia coccoides or use in the treatment or prevention of psoriasis.

In some embodiments, bacterial strains from the species Blautia producta or Blautia coccoides may provide therapeutic benefits in the treatment or prevention of systemic lupus erythematosus (SLE). In certain embodiments, the compositions of the invention are for use in the treatment or prevention of SLE in a subject. In certain embodiments, the invention provides a composition comprising a bacterial strain of the species Blautia producta or Blautia coccoides for use in the treatment or prevention of SLE.

In some embodiments, bacterial strains from the species Blautia producta or Blautia coccoides may provide therapeutic benefits in the treatment or prevention of allograft rejection. In certain embodiments, the compositions of the invention are for use in the treatment or prevention of allograft rejection in a subject. In certain embodiments, the invention provides a composition comprising a bacterial strain of the species Blautia producta or Blautia coccoides for use in the treatment or prevention of allograft rejection.

In some embodiments, the bacterial strain is viable and capable of partially or totally colonising the intestine.

The bacterial strain in the composition is of the species Blautia producta or Blautia coccoides. In preferred embodiments of every aspect of the invention, the composition comprises a bacterial strain of the species Blautia producta. Strains closely related to those tested in the examples may preferably be used, such as bacterial strains that have a 16s rRNA gene sequence that is at least 95%, 96%, 97%, 98%, 99%, 99.5% or 99.9% identical to SEQ ID NO:1, 2 or 3, preferably SEQ OD NO:1. Preferably, the bacterial strain for use in the invention has the 16s rRNA gene sequence represented by SEQ ID NO: 1.

In some embodiments, the composition does not contain any other bacterial strains or species or wherein the composition comprises only de minimis or biologically irrelevant amounts of other bacterial strains or species.

In certain embodiments, the composition of the invention is for oral administration. Oral administration is convenient for patients and practitioners and allows delivery to and/or partial or total colonisation of the intestine.

In certain embodiments, the composition of the invention comprises one or more pharmaceutically acceptable excipients or carriers.

In certain embodiments, the composition of the invention comprises a bacterial strain that has been lyophilised. Lyophilisation is an effective and convenient technique for preparing stable compositions that allow delivery of bacteria.

In certain embodiments, the invention provides a food product comprising the composition as described above.

In developing the above invention, the inventors have identified and characterised a bacterial strain that is particularly useful for therapy. The Blautia producta strain of the invention is shown to be effective for treating the diseases described herein, such as colitis and GVHD. Therefore, in another aspect, the invention provides a cell of the Blautia producta strain deposited under accession number NCIMB 43170, or derivatives thereof. The invention also provides compositions comprising such cells, or biologically pure cultures of such cells. Such compositions may further comprise a pharmaceutically acceptable carrier or excipient. The invention also provides a cell of the Blautia producta strain deposited under accession number NCIMB 43170, or derivatives thereof, for use in therapy, in particular for the diseases described herein.

Further numbered embodiments of the invention are provided below:
1. A composition comprising a bacterial strain of the species Blautia producta or Blautia coccoides, for use in treating or preventing an inflammatory or autoimmune disease.
2. The composition according to embodiment 1, for use in the treatment or prevention of a disease or condition selected from the list consisting of: graft-versus-host disease; inflammatory bowel diseases, such as Crohn's disease or ulcerative colitis; asthma, such as allergic asthma or neutrophilic asthma; arthritis, such as rheumatoid arthritis, osteoarthritis, psoriatic arthritis, or juvenile idiopathic arthritis; multiple sclerosis; psoriasis; systemic lupus erythematosus; and allograft rejection.
3. The composition of embodiment 2, for use in the treatment or prevention of a graft-versus-host disease.
4. The composition according to embodiment 2, wherein the composition is for use in a method of treating or preventing graft-versus-host disease.
5. The composition according to embodiment 4, wherein the composition is for use in reducing weight loss or enhancing weight gain.
6. The composition according to embodiment 4, wherein the composition is for use in reducing protecting skin integrity or improving skin integrity.
7. The composition according to embodiment 4, wherein the composition is for use in reducing gut permeability.
8. The composition according to embodiment 4, wherein the composition is for use in treating intestinal GVHD.
9. The composition according to embodiment 4, wherein the composition is for use in treating skin GVHD.
10. The composition according to embodiment 4, wherein the composition is for use in treating upper gut GVHD.
11. The composition according to embodiment 4, wherein the composition is for use in treating grade III or grade IV acute GVHD.
12. The composition according to embodiment 2, wherein the composition is for use in a method of treating or preventing inflammatory bowel disease.
13. The composition according to embodiment 12, wherein the composition is for use in reducing ulcerations and/or bleeding.
14. The composition according to embodiment 12, wherein the composition is for use in reducing weight loss or enhancing weight gain.
15. The composition according to embodiment 12, wherein the composition is for use in reducing gut permeability.
16. The composition according to embodiment 12, wherein the composition is for use in a patient with GVHD.
17. The composition according to embodiment 2, wherein the composition is for use in a method of treating or preventing asthma.
18. The composition according to embodiment 2, wherein the composition is for use in a method of treating or preventing arthritis.
19. The composition according to embodiment 2, wherein the composition is for use in a method of treating or preventing multiple sclerosis.
20. The composition according to embodiment 2, wherein the composition is for use in a method of treating or preventing psoriasis.
21. The composition according to embodiment 2, wherein the composition is for use in a method of treating or preventing systemic lupus erythematosus.
22. The composition according to embodiment 2, wherein the composition is for use in a method of treating or preventing allograft rejection.
23. The composition of any preceding embodiment, wherein the bacterial strain is viable and capable of partially or totally colonising the intestine.
24. The composition of any preceding embodiment, wherein the composition does not contain any other bacterial strains or species or wherein the composition comprises only de minimis or biologically irrelevant amounts of other bacterial strains or species.
25. The composition of any preceding embodiment, wherein the bacterial strain has a 16s rRNA gene sequence that is at least 95%, 96%, 97%, 98%, 99%, 99.5% or 99.9% identical to SEQ ID NO:1, 2 or 3, preferably SEQ OD NO:1.
26. The composition of any preceding embodiment, wherein the bacterial strain has a 16s rRNA gene sequence represented by SEQ ID NO:1.
27. The composition of any preceding embodiment, wherein the composition is for oral administration.
28. The composition of any preceding embodiment, wherein the composition comprises one or more pharmaceutically acceptable excipients or carriers.
29. The composition of any preceding embodiment, wherein the bacterial strain is lyophilised.
30. A food product comprising the composition of any preceding embodiment, for the use of any preceding embodiment.
31. A cell of the Blautia producta strain deposited under accession number NCIMB 43170, or a derivative thereof.
32. A composition comprising the cell of embodiment 31.
33. The composition of embodiment 32, comprising a pharmaceutically acceptable carrier or excipient.
34. A biologically pure culture of the Blautia producta strain deposited under accession number NCIMB 43170, or a derivative thereof.
35. A cell of the Blautia producta strain deposited under accession number NCIMB 43170, or a derivative thereof, for use in therapy, preferably for use in the treatment or prevention of a disease or condition as defined in one of embodiments 1-22.

### BRIEF DESCRIPTION OF DRAWINGS

FIG. 1 GVHD body weight data in mice models administered strain NCIMB 43170. Animals were weighed daily for the duration of the study, and body weight in g is shown. To determine statistically significant difference between groups, area under the curve (AUC) was calculated using the trapezoidal transformation rule and is shown in figure inset. Significance was determined by one-way ANOVA with Tukey's multiple comparisons test. Asterisk indicates significance as compared to Group 1; hash indicates significance as compared to Group 2; and dot indicates significance as compared to Group 3; unless otherwise indicated. *p<0.05, **p<0.01, ***p<0.005, ****p<0.001. Mean±SEM for each group is shown. n=8-12 per group.

FIG. 2 GVHD body weight data in mice models administered strain NCIMB 43170. Animals were weighed daily for the duration of the study, and percent body weight change relative to Day −14 is shown. Asterisk indicates significance as compared to Group 1; hash indicates significance as compared to Group 2; and dot indicates significance as compared to Group 3; unless otherwise indicated. *p<0.05, **p<0.01, ***p<0.005, ****p<0.001. Data is presented as mean±SEM. n=8-12 per group.

FIG. 3 GVHD body weight data in mice models administered strain NCIMB 43170. Animals were weighed daily for the duration of the study, and percent body weight change relative to Day 0 is shown. Asterisk indicates significance as compared to Group 1; hash indicates significance as compared to Group 2; and dot indicates significance as compared to Group 3; unless otherwise indicated. *p<0.05, **p<0.01, ***p<0.005, ****p<0.001. Data is presented as mean±SEM. n=8-12 per group.

FIG. 4 GVHD body weight data in mice models administered strain NCIMB 43170 accounting for group attrition, the body weight with which an animal died was carried forward for the duration of the study for animals found dead or euthanized for all groups except Group 2. Asterisk indicates significance as compared to Group 1; hash indicates significance as compared to Group 2; and dot indicates significance as compared to Group 3; unless otherwise indicated. *p<0.05, **p<0.01, ***p<0.005, ****p<0.001. Data is presented as mean±SEM. n=8-12 per group.

FIG. 5 GVHD body weight data in mice models administered tacrolimus (FK506) ***: p<0.005.

FIG. 6 Animal survival in mice models administered with strain NCIMB 43170.

FIG. 7 Animal survival in mice models administered with tacrolimus (FK506).

FIG. 8 GVHD clinical scores in mice models administered strain NCIMB 43170. Animals were assigned a clinical GVHD score daily from Days 0 to 30. Area under the curve (AUC) was calculated using the trapezoidal transformation rule and is shown in figure inset. Asterisk indicates significance as compared to Group 1; hash indicates significance as compared to Group 2; and dot indicates significance as compared to Group 3; unless otherwise indicated. *p<0.05, **p<0.01, ***p<0.005, ****p<0.001. Data is presented as mean±SEM. n=8-12 per group.

FIG. 9 GVHD clinical scores in mice models administered strain NCIMB 43170. Animals were assigned a clinical GVHD score daily from Days 0 to 30. To account for group attrition, the GVHD score with which an animal died was carried forward for the duration of the study for animals found dead or euthanized for all groups except Group 2. Area under the curve (AUC) was calculated using the trapezoidal transformation rule and is shown in figure inset. Asterisk indicates significance as compared to Group 1; hash indicates significance as compared to Group 2; and dot indicates significance as compared to Group 3; unless otherwise indicated. *p<0.05, **p<0.01, ***p<0.005, ****p<0.001. Data is presented as mean±SEM. n=8-12 per group.

FIGS. 10A-10E Animals were given a clinical GVHD score daily. The clinical GVHD score is a composite of (FIG. 10A) Posture, (FIG. 10B) Activity, (FIG. 10C) Fur Texture, (FIG. 10D) Skin Integrity, and (FIG. 10E) Weight Loss used in composite GVHD scores in mice models administered strain NCIMB 43170.

FIG. 11 GVHD clinical scores in mice models administered tacrolimus (FK506).

FIG. 12 Colitis severity scores in mice models administered strain NCIMB 43170. Animals underwent video endoscopy on Day 29 to assess colon inflammation. Asterisk indicates significance as compared to Group 1; hash indicates significance as compared to Group 2; and dot indicates significance as compared to Group 3; unless otherwise indicated. *p<0.05, **p<0.01, ***p<0.005, ****p<0.001. Data is presented as mean±SEM. n=8-12 per group.

FIG. 13 Representative colon endoscopy images.

FIG. 14 Plasma citrulline levels in mice administered strain NCIMB 43170. Blood was collected prior to euthanasia from all surviving animals and was processed for plasma; plasma citrulline was assessed in duplicate by ELISA. Plasma was diluted 1:10 for analysis. Asterisk indicates significance as compared to Group 1; hash indicates significance as compared to Group 2; and dot indicates significance as compared to Group 3; unless otherwise indicated. *p<0.05, **p<0.01, ***p<0.005, ****p<0.001. Data is presented as mean±SEM. n=8-12 per group.

FIG. 15 SCFA production by strain NCIMB 43170 and reference strains of Blautia producta and/or Blautia coccoides.

FIG. 16 IL-6 secretion by human U373 cells treated with Blautia coccoides strain A supernatant.

FIG. 17 IL-6 secretion by human U373 cells treated with Blautia coccoides strain B supernatant.

FIG. 18 Acetylated histone H3, acetylated histone H4 and occludin levels in HCT116 colorectal cells treated with Blautia coccoides strain B supernatant.

### DISCLOSURE OF THE INVENTION

### Bacterial Strains

The compositions of the invention comprise a bacterial strain of the species Blautia producta or Blautia coccoides. The examples demonstrate that bacteria of Blautia producta are useful for treating and preventing GVHD and colitis and Blautia coccoides is extremely similar to Blautia producta.

The Blautia species are Gram-reaction-positive, non-motile bacteria that may be either coccoid or oval and all are obligate anaerobes that produce acetic acid as the major end product of glucose fermentation [17]. Blautia producta may be isolated from the human gut. The 16S rRNA gene sequences of the Blautia producta strain used in the examples is disclosed herein as SEQ ID NO:1.

Blautia producta strains are described in [17]. The type strain, ATCC 27340 (=DSM 2950=JCM 1417), was isolated from a human septicaemia sample. This type strain was originally deposited as Ruminoccoccus productus but has since been reclassified as Blautia producta. The GenBank/EMBL/DDBJ accession number for a partial 16S rRNA gene sequence of the Blautia producta type strain ATCC 27340 is AB600998.1 The whole genome sequence is available under accession number ASM37388v1. An additional strain of Blautia producta (Ruminococcus productus) has been deposited under accession number DSM 3508 (ATCC 35244). These strains of Blautia producta are expected to exhibit the same therapeutic effects as the strains tested in the examples.

Blautia coccoides strains are described in [17]. Blautia producta and Blautia coccoides share a very high 16S rRNA gene sequence similarity and are almost identical in terms of their biochemical profiles, products of glucose metabolism and DNA G+C content [17]. Therefore, although in general different bacterial species have different characteristics, strains of Blautia coccoides are expected to exhibit the same therapeutic effects as the strains tested in the examples. The Blautia coccoides type strain is ATCC 29236T (5DSM 935T5JCM 1395T5NCTC 11035T), which was originally deposited as Clostridium coccoides but has since been reclassified as Blautia coccoides.

The Blautia producta bacterium deposited under accession numbers NCIMB 43170 was tested in the Examples and is the preferred strain of the invention. A 16S rRNA gene sequence for the NCIMB 43170 strain that was tested is provided in SEQ ID NO:1. Strain NCIMB 43170 was deposited with the international depositary authority NCIMB, Ltd. (Ferguson Building, Craibstone Estate, Bucksburn, Aberdeen, AB21 9YA, Scotland) by 4D Pharma Research Limited (Life Sciences Innovation Building, Aberdeen, AB25 2ZS, Scotland) on 20 Aug. 2018 as “Blautia producta” and was assigned accession number NCIMB 43170.

Additional reference Blautia producta/Blautia coccoides strains were also tested in Example 2. It was not possible to definitively classify the reference strains as belonging to Blautia producta or Blautia coccoides using 16S sequencing and MALDI-TOF MS analysis, due to the unusually high similarity between these two species. All of the reference strains were found to produce acetate and to not produce butyrate or propionate. 16S rRNA gene sequences for strains REF1 and REF2 that were tested are provided in SEQ ID NO:2 and SEQ ID NO:3.

Bacterial strains closely related to the strain tested in the Examples are also expected to be effective for treating or preventing other inflammatory and autoimmune diseases. In certain embodiments, the bacterial strain for use in the invention has a 16s rRNA gene sequence that is at least 95%, 96%, 97%, 98%, 99%, 99.5% or 99.9% identical to SEQ ID NO:1, 2 or 3, preferably SEQ OD NO:1. Preferably, the bacterial strain for use in the invention has the 16s rRNA gene sequence represented by SEQ ID NO:1.

Bacterial strains that are biotypes of the bacterium deposited under accession number NCIMB 43170 are also expected to be effective for treating or preventing GVHD and other inflammatory or autoimmune diseases. A biotype is a closely related strain that has the same or very similar physiological and biochemical characteristics.

Strains that are biotypes of a bacterium deposited under accession number NCIMB 43170 and that are suitable for use in the invention may be identified by sequencing other nucleotide sequences for a bacterium deposited under accession numbers NCIMB 43170. For example, substantially the whole genome may be sequenced and a biotype strain for use in the invention may have at least 95%, 96%, 97%, 98%, 99%, 99.5% or 99.9% sequence identity across at least 80% of its whole genome (e.g. across at least 85%, 90%, 95% or 99%, or across its whole genome). Other suitable sequences for use in identifying biotype strains may include hsp60 or repetitive sequences such as BOX, ERIC, (GTG)₅, or REP [18]. Biotype strains may have sequences with at least 95%, 96%, 97%, 98%, 99%, 99.5% or 99.9% sequence identity to the corresponding sequences of a bacterium deposited under accession number NCIMB 43170.

Alternatively, strains that are biotypes of a bacterium deposited under accession number NCIMB 43170 and that are suitable for use in the invention may be identified by using the accession number NCIMB 43170, and restriction fragment analysis and/or PCR analysis, for example by using fluorescent amplified fragment length polymorphism (FAFLP) and repetitive DNA element (rep)-PCR fingerprinting, or protein profiling, or partial 16S or 23s rDNA sequencing. In preferred embodiments, such techniques may be used to identify other Blautia strains.

In certain embodiments, strains that are biotypes of a bacterium deposited under accession number NCIMB 43170 and that are suitable for use in the invention are strains that provide the same pattern as a bacterium deposited under accession number NCIMB 43170 when analysed by amplified ribosomal DNA restriction analysis (ARDRA), for example when using Sau3AI restriction enzyme (for exemplary methods and guidance see, for example, [19]. Alternatively, biotype strains are identified as strains that have the same carbohydrate fermentation patterns as a bacterium deposited under accession numbers NCIMB 43170.

Other Blautia producta and Blautia coccoides strains that are useful in the compositions and methods of the invention, such as biotypes of a bacterium deposited under accession number NCIMB 43170, may be identified using any appropriate method or strategy, including the assays described in the examples. In particular, bacterial strains that have similar growth patterns, metabolic type and/or surface antigens to a bacterium deposited under accession number NCIMB 43170 may be useful in the invention. A useful strain will have comparable immune modulatory activity to strain NCIMB 41370. In particular, a biotype strain will elicit comparable effects on GVHD and colitis as shown in the examples, which may be identified by using the culturing and administration protocols described in the examples.

A particularly preferred strain of the invention is the Blautia producta strain deposited under accession number NCIMB 43170. This is the exemplary Blautia producta strain tested in the examples and shown to be effective for treating GVHD and colitis. Therefore, the invention provides a cell, such as an isolated cell, of the Blautia producta strain deposited under accession number NCIMB 43170, or a derivative thereof. The invention also provides a composition comprising a cell of the Blautia producta strain deposited under accession number NCIMB 43170, or a derivative thereof. The invention also provides a biologically pure culture of the Blautia producta strain deposited under accession number NCIMB 43170. The invention also provides a cell of the Blautia producta strain deposited under accession number NCIMB 43170, or a derivative thereof, for use in therapy, in particular for the diseases described herein.

A derivative of the strain deposited under accession number NCIMB 43170 may be a daughter strain (progeny) or a strain cultured (subcloned) from the original. A derivative of a strain of the invention may be modified, for example at the genetic level, without ablating the biological activity. In particular, a derivative strain of the invention is therapeutically active. A derivative of the NCIMB 43170 strain will generally be a biotype of the NCIMB 43170 strain.

The bacterium deposited under accession number NCIMB 43170 does not produce butyrate but is shown to be effective for treating disease in the examples. Therefore, in preferred embodiments, the composition comprises a bacterial strain of the species that does not produce butyrate. Additionally or alternatively, the bacterial compositions of the invention do not comprise bacterial strains which individually and/or collectively produce butyrate. Butyrate production may be measured using any appropriate method available in the art, including those of Example 2. Thus, in certain embodiments, the composition of the invention comprises a bacterial strain of the species that does not produce butyrate, or the bacterial compositions of the invention do not comprise bacterial strains which individually and/or collectively produce butyrate, when cultured for 16 hours using YCFA or PYG media. In certain embodiments, butyrate production is measured following inoculating YCFA or PYG media with 10% inoculum, which has been pre-prepared and pre-equilibrated at least 24 hours prior. In certain embodiments, measurements of less than 1 mM in the assay of Example 2 are considered to indicate that a bacterial strain does not produce butyrate.

In certain embodiments, the composition comprises a bacterial strain that does not produce propionate. The bacterium deposited under accession number NCIMB 43170 and the other B. producta/Blautia coccoides strains tested in the examples do not produce propionate. Propionate production may be measured using any appropriate method available in the art, including those of Example 2. In certain embodiments, the composition comprises a bacterial strain that produces acetate, for example at 20-40 mM, for example using the method of Example 2. The bacterium deposited under accession number NCIMB 43170 and the other B. producta/Blautia coccoides strains tested in the examples produce acetate. Acetate production may be measured using any appropriate method available in the art, including those of Example 2. In preferred embodiments of the invention the composition comprises a bacterial strain that does not produce butyrate or propionate. In preferred embodiments of the invention the composition comprises a bacterial strain that does not produce butyrate and does produce acetate. In preferred embodiments of the invention the composition comprises a bacterial strain that does not produce butyrate or propionate and does produce acetate. In certain embodiments, the composition comprises a bacterial strain that produces propanoic acid, 2-methyl-propanoic acid (isobutyric acid), 3-methyl-butanoic acid (isovaleric acid) and pentanoic acid when grown in YCFA.

References to cells of the Blautia producta strain deposited under accession number NCIMB 43170 encompass any cells that have the same safety and therapeutic efficacy characteristics as the strains deposited under accession number NCIMB 43170, and such cells are encompassed by the invention.

In preferred embodiments, the bacterial strains in the compositions of the invention are viable and capable of partially or totally colonising the intestine.

### Therapeutic Uses

As demonstrated in the examples, the bacterial compositions of the invention are effective for treating GVHD and colitis associated with GVHD. GVHD is a prototypical inflammatory or autoimmune disease, so the compositions of the invention may be effective for reducing inflammation and treating inflammatory and autoimmune diseases. In preferred embodiments, the compositions of the invention are for use in treating a disease selected from the list consisting of: graft-versus-host disease (GVHD); inflammatory bowel diseases, such as Crohn's disease or ulcerative colitis; asthma, such as allergic asthma or neutrophilic asthma; arthritis, such as rheumatoid arthritis, osteoarthritis, psoriatic arthritis, or juvenile idiopathic arthritis; multiple sclerosis; psoriasis; systemic lupus erythematosus; and allograft rejection.

GVHD occurs following allogenic tissue or stem cell transplantation. Donor T cells in the transplanted tissue recognise host peptides as foreign and differentiate into cytokine-producing T effector cells. This leads to the proinflammatory cytokine cascade that is characteristic of acute GVHD [20]. Acute GVHD generally affects the skin, liver and intestinal tract. Acute GVHD may progress to a chronic GVHD, which may extend to the lung, eyes and mucous membranes and has clinical features that resemble those of autoimmune diseases [21].

GVHD is mediated by donor-derived T cells. The donor-derived naïve CD4⁺ T cells become activated by antigens expressed on host tissues, and differentiate into Th-cell subsets of effector T cells. It has been shown that wild type T cells predominately differentiate into Th1 cells during GVHD and that blockade of differentiation of T cells to Th1 and Th17 cells can ameliorate GVHD in mice [22]. Th1 and Th17 cells are both producers of pro-inflammatory cytokines. Th17 cells produce IL-17 and IL-21 and IL-22 cytokines. Th1 cells produce IFN-γ and IL-2 cytokines. The Th1 and Th17 pathways have the capacity to cause autoimmune disease independently or collaboratively (as described in, for example, [23-31], so the compositions of the invention may be useful for treating inflammatory and autoimmune diseases.

The model tested in the examples may be particularly relevant to acute GVHD because GVHD pathology developed quickly in the mice. Therefore, the compositions of the invention may be particularly useful for treating or preventing acute diseases or conditions as listed above. In certain embodiments, the compositions of the invention are for use in treating an acute inflammatory or autoimmune disease. In certain embodiments, the patient may have been diagnosed with an acute inflammatory or autoimmune disease or condition.

GVHD, as studied in the examples, may be chronic, so the compositions of the invention may be particularly useful for treating or preventing chronic diseases or conditions as listed above. In certain embodiments, the compositions of the invention are for use in treating a chronic inflammatory or autoimmune disease. In certain embodiments, the patient may have been diagnosed with a chronic inflammatory or autoimmune disease or condition, or the composition of the invention may be for use in preventing an inflammatory or autoimmune disease or condition developing into a chronic inflammatory or autoimmune disease or condition.

In certain embodiments, treatment with compositions of the invention provides a reduction or prevents an elevation in IL-17, IL-21 or IL-22 levels. In certain embodiments, treatment with compositions of the invention provides a reduction or prevents an elevation in TNFα, IFN-γ, or IL-6 levels. Such reduction or prevention of elevated levels of these cytokines may be useful for treating or preventing inflammatory and autoimmune diseases and conditions.

In certain embodiments, treatment with compositions of the invention provides a reduction or prevents an elevation in IL-2 or IFN-γ levels. Such reduction or prevention of elevated levels of these cytokines may be useful for treating or preventing inflammatory and autoimmune diseases and conditions.

Evidence from germ-free animal studies has indicated that the development and function of the intestinal barrier are dependent on the gut microbiota. Many inflammatory and autoimmune diseases have been shown to be associated with increased intestinal permeability (“leaky gut”), including inflammatory bowel diseases, such as Crohn's disease or ulcerative colitis, asthma, arthritis, multiple sclerosis, psoriasis and systemic lupus erythematosus [32-35). It has been suggested that the autoimmune process can be arrested by re-establishment of the intestinal barrier function [36]. The examples demonstrate that treatment with compositions of the invention can lead to a decrease in intestinal permeability, which may be useful for treating or preventing inflammatory and autoimmune diseases and conditions. In certain embodiments, the compositions of the invention are for reducing intestinal permeability in the treatment or prevention of an inflammatory or autoimmune disease. In certain embodiments, the compositions of the invention are for use in treating an inflammatory or autoimmune disease associated with increased intestinal permeability, or are for use in treating an inflammatory or autoimmune disease in a patient diagnosed as exhibiting increased intestinal permeability.

Strikingly, the examples demonstrate that the compositions of the invention may affect disease processes distal from the gastrointestinal tract, such as GVHD. In certain embodiments, the invention provides a composition comprising a bacterial strain of the species Blautia producta or Blautia coccoides for use in the treatment or prevention of an inflammatory or autoimmune disease that is distal from the gastrointestinal tract. In certain embodiments, the invention provides a composition comprising a bacterial strain of the species Blautia producta or Blautia coccoides for use in the treatment or prevention of an inflammatory or autoimmune disease that is distal from the gastrointestinal tract. In certain embodiments, the compositions of the invention are for use in treating disease that is not bowel disease, such as is not inflammatory bowel disease. In certain embodiments, the compositions of the invention are for use in treating an inflammatory or autoimmune disease that does not affect the gastrointestinal tract. In certain embodiments, the compositions of the invention are for reducing inflammation of a tissue that is not part of the gastrointestinal tract.

In certain embodiments, the compositions of the invention are for treating an autoimmune or inflammatory disease that is not associated with IL-17 or the Th17 pathway. In certain embodiments, the compositions of the invention are for treating an autoimmune or inflammatory disease in a patient that does not have elevated 11-17 levels.

In embodiments of the invention, the compositions of the invention are for treating grade III or grade IV GVHD.

In certain embodiments of the invention, the compositions of the invention are for treating skin GVHD or upper gut GVHD.

### Graft-Versus-Host Disease (GVHD)

The compositions of the invention may be for use in the treatment or prevention of graft-versus-host disease (GVHD). GVHD is a medical complication following transplantation of allogeneic tissue into a subject. GVHD commonly occurs following stem cell or bone marrow transplantation or solid organ transplantation, particularly where the genetic background of the graft (i.e. the donor) and the host (i.e. the recipient) are distinct.

The pathophysiology of GVHD comprises three distinct phases. Firstly, host antigen presenting cells (APCs), such as dendritic cells (DCs) are activated following recognition of the transplanted tissue as a foreign substance. APC activation precedes the recruitment and activation of effector immune cells, such as conventional cytotoxic T cells, which leads to destruction or rejection of the foreign tissue.

GVHD differs significantly from allograft rejection in a number of aspects, including the pathways by which antigen-presenting cells are stimulated, the tissue damage that occurs, the role of different cytokines, the role of lymphoid cells, the role of the microbiome and the role of Notch signalling (see [37] for review).

In certain embodiments, the compositions of the invention may be administered after the patient has received the transplant. In certain embodiments, the compositions of the invention may be administered before the patient has received the transplant. In certain embodiments, the compositions of the invention may be administered to the donor before the transplant. Administration of the compositions of the invention before the transplant has been received may be useful in priming the immune system of the patient or donor to not elicit an inflammatory or autoimmune response. In certain embodiments, the compositions of the invention may be used for preventing or preventing the onset of GVHD. In certain embodiments, the composition of the invention may be for use in the treatment or prevention of GVHD prophylactically.

In certain embodiments, the compositions of the invention may be useful for treating, delaying, preventing, or preventing the onset of acute GVHD. Symptoms of acute GVHD typically manifest within the first 100 days of transplantation. Delaying, treatment or prevention of acute GVHD may be particularly beneficial to aid the recovery of subjects in the immediate aftermath of transplant surgery.

In certain embodiments, the compositions of the invention are for use in preventing death or improving survival following transplant surgery, such as stem cell or bone marrow surgery.

In certain embodiments, the compositions of the invention may treat, delay the onset of, prevent, or prevent the onset of acute GVHD when administered to a subject within 100 days following transplantation. In certain embodiments, the compositions of the invention may treat, delay the onset of, prevent, or prevent the onset of acute GVHD when administered to a subject prophylactically, for example, when the composition is administered to the subject before the transplant. In certain embodiments, the compositions of the invention may treat, delay the onset of, prevent, or prevent the onset of persistent, late-onset or recurrent acute GVHD, such as acute GVHD that occurs or recurs more than 100 days after transplantation.

In certain embodiments, the composition of the invention may treat, delay the onset of, prevent, or prevent the onset one or more symptoms of acute GVHD selected from the list consisting of macropaular skin rash, nausea, anorexia, diarrhea, severe abdominal pain, ileus and cholestatic hyperbilirubinemia.

The examples demonstrate that the compositions of the invention are effective for reducing weight loss, skin damage and gut permeability associated with GVHD. Therefore, in certain embodiments, the compositions of the invention are for use in reducing weight loss or enhancing weight gain in the treatment of GVHD. In certain embodiments, the compositions of the invention are for use in protecting skin integrity or improving skin integrity in the treatment of GVHD. In certain embodiments, the compositions of the invention are for use in reducing gut permeability in the treatment GVHD. In preferred embodiments, the compositions of the invention are for use in treating intestinal GVHD. In preferred such embodiments, the composition of the invention comprises a strain of the species Blautia Producta.

In certain embodiments, the compositions of the invention may be useful for treating, delaying the onset of, preventing, or preventing the onset of chronic GVHD. Chronic GVHD is a complex, multisystem disorder that can involve any organ and is typically characterised by fibrosis. Chronic GVHD may evolve from acute GVHD, or may emerge after a period of quiescence following acute GVHD, or may emerge de novo. Symptoms of chronic GVHD may emerge at any time following transplantation. The compositions may be useful for treating, preventing, preventing the onset of, or delaying the onset of chronic GVHD by reducing or preventing elevation of the Th17 and/or Th1 inflammatory response. The compositions may be useful for treating, preventing, preventing the onset of, or delaying the onset of chronic GVHD by inhibiting HDAC activity. The compositions may treat, delay the onset of, prevent, or prevent the onset of chronic GVHD by upregulating Treg cell activity. The compositions may treat, delay the onset of, prevent, or prevent the onset of chronic GVHD by inhibiting conventional cytotoxic T cell activity. The compositions of the invention may treat, delay the onset of, prevent, or prevent the onset of chronic GVHD by enhancing NK cell activity. The compositions of the invention may treat, delay the onset of, prevent, or prevent the onset of chronic GVHD by inhibiting APC DC activation.

In certain embodiments, the compositions of the invention are for administration to a patient that has recently undergone a stem cell, bone marrow or solid organ transplant. In certain embodiments, the compositions of the invention are for administration to a patient is in need of a stem cell, bone marrow or solid organ transplant.

In certain embodiments, the composition of the invention may treat, delay the onset of, prevent, or prevent the onset of one or more symptoms of chronic GVHD selected from the list consisting of: dyspigmentation, new-onset alopecia, poikiloderma, lichen planus like eruptions or sclerotic features, nail dystrophy or loss, xerostomia, mouth ulcers (such as aphthous stomatitis), lichen-type features in the mouth (such as lichen sclerosis), keratoconjunctivitis sicca, sicca syndrome, cicatricial conjunctivitis, fascititis, myostitis, joint stiffness, vaginal sclerosis, ulcerations, anorexia, weight loss, oesophageal web, jaundice, transaminitis, pleural effusions, bronchiolitis obliterans, nephrotic syndrome, pericarditis, thrombocytopenia, anemia, and neutropenia.

In certain embodiments, the compositions of the invention may be for use in combination with one or more pharmacological agents for the treatment or prevention of GVHD. In certain embodiments, the one or more pharmacological agents are for the pharmacological prevention or treatment of GVHD. In certain embodiments, the compositions of the invention are for use in the treatment or prevention of GVHD in a subject who is receiving, has received, or is about to receive, one or more of said pharmacological agents. In certain embodiments, the one or more pharmacological agents are selected from the list consisting of: suberoylanilide, vorisnostat, ITF2357 cyclosporine, ciclosporin, sirolimus, pentostatin, rituximab, imatinib, mycophenolate mofetil, tacrolimus, prednisone, methotrexate, remestemcel-L and Prochymal, wherein the pharmacological agent is administered in a therapeutically effective amount for the treatment or prevention of GVHD. In some embodiments, the compositions of the invention are for use in the treatment of GVHD in a subject who has received, is receiving, or is about to receive extracorporeal photophoreses.

In preferred embodiments, the compositions of the invention are for use in treating GVHD and comprise a single strain of Blautia producta and no other bacterial strains or species, or only de minimis or biologically irrelevant amounts of other bacterial strains or species.

In preferred embodiments, the compositions of the invention are for use in treating GVHD and the bacterial strain does not produce butyrate. In preferred embodiments, the compositions of the invention are for use in treating GVHD and do not comprise any bacterial strains or species that produce butyrate.

In further preferred embodiments, the compositions of the invention are for use in treating GVHD and comprise a single strain of Blautia producta that does not produce butyrate and no other bacterial strains or species, or only de minimis or biologically irrelevant amounts of other bacterial strains or species.

In preferred embodiments, the compositions of the invention are for use in treating GVHD in a patient that has not received an antibiotic, such as has not received an antibiotic in the preceding day, week or month. In preferred embodiments, the compositions of the invention are for use in treating GVHD and are to be administered as a monotherapy. In further preferred embodiments, the compositions of the invention are for use as a monotherapy in treating GVHD and comprise a single strain of Blautia producta and no other bacterial strains or species, or only de minimis or biologically irrelevant amounts of other bacterial strains or species. In further preferred embodiments, the compositions of the invention are for use as a monotherapy in treating GVHD and do not comprise any bacterial strains or species that produce butyrate. In particularly preferred embodiments, the compositions of the invention are for use as a monotherapy in treating GVHD and comprise a single strain of Blautia producta that does not produce butyrate and no other bacterial strains or species, or only de minimis or biologically irrelevant amounts of other bacterial strains or species.

### Inflammatory Bowel Disease

The examples demonstrate that the compositions of the invention can reduce the severity of colitis, and so they may be useful in the treatment of inflammatory bowel diseases.

In certain embodiments, the compositions of the invention are for use in treating or preventing inflammatory bowel disease.

Inflammatory bowel disease (IBD) is a complex disease that can be caused by multiple environmental and genetic factors. Factors contributing to the onset of IBD include diet, microbiota, intestinal permeability, and genetic susceptibility to increased inflammatory response to gut infection. Symptoms of inflammatory bowel disease include abdominal pain, vomiting, diarrhoea, rectal bleeding, severe internal cramps/muscle spasms in the pelvic region, weight loss and anaemia. In certain embodiments, the compositions are for use in reducing one or more symptoms associated with IBD. In certain embodiments, the compositions of the invention are for use in preventing one or more symptoms of IBD.

The examples demonstrate that the compositions of the invention may reduce the severity of colitis, and in particular may reduce ulceration and bleeding. In certain embodiments, the compositions of the invention are for use in reducing or preventing ulceration or bleeding in the treatment of an inflammatory bowel disease. The examples also demonstrate that the compositions of the invention are effective for reducing weight loss and gut permeability associated with colitis and GVHD. Therefore, in certain embodiments, the compositions of the invention are for use in reducing weight loss or enhancing weight gain in the treatment of an inflammatory bowel disease. In certain embodiments, the compositions of the invention are for use in reducing gut permeability in the treatment an inflammatory bowel disease. In preferred embodiments, the composition of the invention comprises a strain of the species Blautia producta.

The examples demonstrate that the compositions of the invention are effective for treating colitis associated with GVHD. Therefore, in certain embodiments, the compositions of the invention are for use in treating colitis in a patient with GVHD. Therefore, in certain embodiments, the compositions of the invention are for use in treating inflammatory bowel disease in a patient with GVHD. In certain embodiments, the compositions are for use in reducing bowel inflammation in the treatment of GVHD.

IBD may accompany other diseases or conditions, such as arthritis, pyoderma gangrenosum, primary sclerosing cholangitis, non-thyroidal illness syndrome, deep vein thrombosis, bronchiolitis obliterans organizing pneumonia. In certain embodiments, the compositions of the invention are for use in the treatment or prevention of one or more diseases or conditions that accompany IBD.

Inflammatory bowel disease is generally diagnosed by biopsy or colonoscopy. Measurements of faecal calprotectin is useful for the preliminary diagnosis of IBD. Other laboratory test for the diagnosis of IBD include, complete blood count, erythrocyte sedimentation rate, comprehensive metabolic panel, faecal occult blood test or C-reactive protein test. Typically a combination of laboratory testing and biopsy/colonoscopy will be used to confirm diagnosis of IBD. In certain embodiments, the compositions of the invention are for use in a subject diagnosed with IBD.

In certain embodiments, the inflammatory bowel disease is ulcerative colitis. Ulcerative colitis is an autoimmune inflammatory bowel disease characterised by infiltrating T cells.

Ulcerative colitis is usually restricted to the rectum and colon but sometimes involves the ileum. The disease is classified depending on the extent of involvement of the gastrointestinal tract. Classifications of ulcerative colitis include distal colitis, such as proctitis, proctosigmoiditis and left-sided colitis, or extensive colitis, such as pancolitis. In certain embodiments, the compositions are for use in the treatment of distal colitis. In certain embodiments, the compositions are for use in the treatment of proctitis. In certain embodiments, the compositions are for use in the treatment of proctosigmoiditis. In certain embodiments, the compositions are for use in the treatment of left-sided colitis. In certain embodiments, the compositions are for use in the treatment of extensive colitis. In certain embodiments, the compositions are for use in the treatment of pancolitis. In certain embodiments, the compositions are for use in the prevention of ulcerative colitis in a subject at risk of developing ulcerative colitis.

Ulcerative colitis is diagnosed by a combination of laboratory testing and surgery, such as endoscopy/colonoscopy and biopsy. Exemplary laboratory test that aid ulcerative colitis diagnosis include complete blood count, complete metabolic panel, liver function tests, urinalysis, stool culture, erythrocyte sedimentation rate and C-reactive protein measurement.

The severity of symptoms of ulcerative colitis can be determined using the Simple Clinical Colitis Activity Index (SCCAI) [38]. SCCAI can also be used as a means to assess efficacy of therapies designed to treat or prevent ulcerative colitis. SCCAI poses the following series of questions designed to determine the severity of ulcerative colitis symptoms: frequency of bowel movements (by day); frequency of bowel movements (by night); urgency of defecation; blood in stool; general well-being; extra-colonic features (for example, arthritis, uveitis, or other conditions that accompany UC). Each answer is provided on a sliding scale generating a score of between 0 and 19. A score of above 5 is usually indicative of the presence of ulcerative colitis.

In some embodiments, the composition is for use in a subject who has been diagnosed with ulcerative colitis. In some embodiments, the compositions are for use in alleviating or ameliorating one or more symptoms of ulcerative colitis. For example, the compositions may improve the score of one or more answers to the SCCAI. In certain embodiments, the compositions of the invention may be for use in reducing the frequency of bowel movements. In certain embodiments, the compositions of the invention may be for use in reducing urgency of defecation. In certain embodiments, the compositions of the invention may be for use in reducing blood in stool. In certain embodiments, the compositions of the invention may be for use in reducing extra-colonic features. The alleviation or amelioration of these symptoms may be determined by an improvement in the corresponding SCCAI score pre- and post-administration of a composition of the invention.

Additional symptoms of ulcerative colitis include diarrhoea, rectal bleeding, weight loss and anaemia, abdominal pain, abdominal cramping with bowel movements. In some embodiments, the compositions of the invention are for use in the treatment or prevention of one or more additional symptoms of ulcerative colitis.

In some instances, ulcerative colitis is accompanied by one or more extra-colonic features. Extra-colonic features are conditions or diseases that accompany ulcerative colitis and manifest outside the colon. Examples of extra-colonic features of ulcerative colitis include: aphthous ulcers, iritis, uveitis, episcleritis, seronegative arthritis, ankylosing spondylitis, sacroiliitis, erythema nodosum, pyoderma grangrenosum, deep venous thrombosis and pulmonary embolism, autoimmune haemolytic anaemia, clubbing, primary sclerosing cholangitis. In some embodiments, the compositions of the invention are for use in treating or preventions one or more extra-colonic features of ulcerative colitis.

Ulcerative colitis may be treated with a number of therapeutics agents, such as 5-aminosalicylic acids, such as sulfasalazine and mesalazine, corticosteroids, such as prednisone, immunosuppressive agents, such as azathioprine, biologics, such as infliximab, adalimumab, and golimumab, vedolizumab and etrolizumab, nicotine, or iron. In certain embodiments, the compositions of the invention are for in the treatment or prevention of ulcerative colitis in combination with an additional therapeutic agent, wherein the additional therapeutic agent is for the treatment or prevention of ulcerative colitis.

In certain embodiments, the compositions of the invention are for use in the treatment or prevention of Crohn's disease.

Crohn's disease is a complex disease with an array of probable causes, including genetic risk factors, diet, other lifestyle factors, such as smoking and alcohol consumption, and microbiome composition. Crohn's disease can manifest anywhere along the gastrointestinal tract.

Gastrointestinal symptoms of Crohn's disease range from mild to severe and include abdominal pain, diarrhoea, faecal blood, ileitis, increased bowel movements, increased flatulence, intestinal stenosis, vomiting, and perianal discomfort. The compositions of the invention may be for use in the treatment of prevention of one or more gastrointestinal symptoms of Crohn's disease.

Systemic symptoms of Crohn's disease include growth defects, such as the inability to maintain growth during puberty, decreased appetite, fever and weight loss. Extra-intestinal features of Crohn's disease include uveitis, photobia, episcleritis, gall stones, seronegative spondyloarthropathy, arthritis, enthesitis, erythema nodosum, pyoderma gangrenosum, deep venous thrombosis, pulmonary embolism, autoimmune haemolytic anaemia, clubbing and osteoporosis. Extra-intestinal features are additional conditions associated with Crohn's disease that manifest outside the GI tract. Subjects with Crohn's disease also exhibit increased susceptibility to neurological complications such as seizures, strokes, myopathy, peripheral neuropathy, headache and depression. In certain embodiments, the compositions of the invention are for use in the treatment or prevention of one or more systemic symptoms of Crohn' disease. In certain embodiments, the compositions of the invention are for use in the treatment or prevention of one or more extra-intestinal features of Crohn's disease.

The diagnosis of Crohn's disease usually involves carrying out multiple tests and surgical procedures, such as gastroscopy and/or colonoscopy and biopsy, typically of the ileum, radiologic tests, complete blood counts, C-reactive protein tests and erythrocyte sedimentation rates. In certain embodiments, the compositions of the invention are for use in subjects diagnosed with Crohn's disease. In some embodiments, compositions of the invention are for use in treating a subject who has been diagnosed with Crohn's disease.

Crohn's disease is classified depending on the extent of the region of the GI tract affected [39]. A disease of both the ileum and colon is classified as Ileocolic Crohn's. In some embodiments, the compositions are for use in the treatment or prevention of Ileocolic Crohn's. In some embodiments, the compositions are for use in a subject diagnosed with Ileocolic Crohn's/Crohn's ileitis is classified if only the ileum is affected. Crohn's colitis is classified if only the colon is affected. In certain embodiments, the compositions are for use in the treatment or prevention of Crohn's ileitis. In some embodiments, the compositions are for use in a subject diagnosed with Crohn's ileitis. In certain embodiments, the compositions are for use in the treatment or prevention of Crohn's colitis. In some embodiments, the compositions are for use in a subject diagnosed with Crohn's colitis.

Crohn's disease may be treated with a number of therapeutic agents, such as corticosteroids, such as prednisone, immunosuppressive agents, such as azathioprine, or biologics, such as infliximab, adalimumab, and golimumab, vedolizumab and etrolizumab. In certain embodiments, the compositions of the invention are for use in the treatment or prevention of Crohn's disease in combination with an additional therapeutic agent. In certain embodiments, the additional therapeutic agent is for use in the treatment or prevention of Crohn's disease.

Irritable bowel syndrome may present with similar symptoms to inflammatory bowel disease. However, irritable bowel syndrome (IBS) is not an example of an inflammatory bowel disease. Notably, the pathogenesis and disease mechanisms underlying IBS and inflammatory bowel disease have very little in common. In particular, inflammatory bowel disease and colitis are driven by chronic intestinal inflammation, whilst in contrast IBS is considered a “functional bowel disease”, which is characterised by the absence of obvious anatomic or physiologic abnormalities [40,41]. In certain embodiments, the compositions of the invention are for use in treating a disease that is not irritable bowel syndrome.

### Asthma

In preferred embodiments, the compositions of the invention are for use in treating or preventing asthma. Asthma is a chronic disease characterised by inflammation and restriction of the airways and the inflammation in asthma may be mediated by Th17 and/or Th1 cells [42,43] and so the compositions of the invention may be particularly effective for preventing or treating asthma. Also, GVHD can result in pulmonary disease affecting the lungs and symptoms of GVHD may include shortness of breath and dry cough. The inflammation in asthma may be mediated by eosinophils and/or neutrophils.

In certain embodiments, the asthma is eosinophilic or allergic asthma. Eosinophilic and allergic asthma are characterised by increased numbers of eosinophils in peripheral blood and in airway secretions and is associated pathologically with thickening of the basement membrane zone and pharmacologically by corticosteroid responsiveness [44]. Compositions that reduce or inhibit eosinophil recruitment or activation may be useful for treating or preventing eosinophilic and allergic asthma.

In additional embodiments, the compositions of the invention are for use in treating or preventing neutrophilic asthma (or non-eosinophilic asthma). High neutrophil numbers are associated with severe asthma that may be insensitive to corticosteroid treatment. Compositions that reduce or inhibit neutrophil recruitment or activation may be useful for treating or preventing neutrophilic asthma.

Eosinophilic and neutrophilic asthma are not mutually exclusive conditions and treatments that help address either the eosinophil and neutrophil responses may be useful for treating asthma in general.

Activation of the Th17 pathway are associated with severe asthma, so the compositions of the invention may be useful for preventing the development of severe asthma or for treating severe asthma.

In certain embodiments, the compositions of the invention are for use in methods reducing an eosinophilic inflammatory response in the treatment or prevention of asthma, or for use in methods of reducing a neutrophilic inflammatory response in the treatment or prevention of asthma. As noted above, high levels of eosinophils in asthma is associated pathologically with thickening of the basement membrane zone, so reducing eosinophilic inflammatory response in the treatment or prevention of asthma may be able to specifically address this feature of the disease. Also, elevated neutrophils, either in combination with elevated eosinophils or in their absence, is associated with severe asthma and chronic airway narrowing. Therefore, reducing the neutrophilic inflammatory response may be particularly useful for addressing severe asthma.

In certain embodiments, the compositions reduce peribronchiolar infiltration in allergic asthma, or are for use in reducing peribronchiolar infiltration in the treatment of allergic asthma. In certain embodiments, the compositions reduce peribronchiolar and/or perivascular infiltration in neutrophilic asthma, or are for use in reducing peribronchiolar and/or perivascular infiltration in the treatment of allergic neutrophilic asthma.

In certain embodiments, treatment with compositions of the invention provides a reduction or prevents an elevation in TNFα levels.

In certain embodiments, the compositions of the invention are for use in a method of treating asthma that results in a reduction of the eosinophilic and/or neutrophilic inflammatory response. In certain embodiments, the patient to be treated has, or has previously been identified as having, elevated neutrophil or eosinophil levels, for example as identified through blood sampling or sputum analysis.

The compositions of the invention may be useful for preventing the development of asthma in a new-born when administered to the new-born, or to a pregnant woman. The compositions may be useful for preventing the development of asthma in children. The compositions of the invention may be useful for treating or preventing adult-onset asthma. The compositions of the invention may be useful for managing or alleviating asthma. The compositions of the invention may be particularly useful for reducing symptoms associated with asthma that is aggravated by allergens, such as house dust mites.

Treatment or prevention of asthma may refer to, for example, an alleviation of the severity of symptoms or a reduction in the frequency of exacerbations or the range of triggers that are a problem for the patient.

### Arthritis

In preferred embodiments, the compositions of the invention are for use in treating or preventing rheumatoid arthritis (RA). Reduction in the percentages of Th1 and Th17 cells and the expression of Th1 and Th17 cytokines is associated with treatment of RA with tocilizumab [45]. Similarly, T cell vaccination leading to the inhibition of Th1 and Th17 could delay the onset of collagen-induced arthritis (an animal model for RA) and reduce joint inflammation [46]. RA is a systemic inflammatory disorder that primarily affects joints. RA is associated with an inflammatory response that results in swelling of joints, synovial hyperplasia, and destruction of cartilage and bone. IL-17 and Th17 cells may have a key role in RA, for example because IL-17 inhibits matrix production in chondrocytes and osteoblasts and activates the production and function of matrix metalloproteinases and because RA disease activity is correlated to IL-17 levels and Th-17 cell numbers [47,48], so the compositions of the invention may be particularly effective for preventing or treating RA.

In certain embodiments, treatment with the compositions of the invention results in a reduction in the swelling of joints. In certain embodiments, the compositions of the invention are for use in patients with swollen joints or patients identified as at risk of having swollen joints. In certain embodiments, the compositions of the invention are for use in a method of reducing joint swelling in RA.

In certain embodiments, treatment with the compositions of the invention results in a reduction in cartilage damage or bone damage. In certain embodiments, the compositions of the invention are for use in reducing or preventing cartilage or bone damage in the treatment of RA. In certain embodiments, the compositions are for use in treating patient with severe RA that are at risk of cartilage or bone damage.

Increased IL-17 levels and Th17 cell numbers are associated with cartilage and bone destruction in RA [49]. IL-17 is known to activate matrix destruction in cartilage and bone tissue and IL-17 has an inhibitory effect on matrix production in chondrocytes and osteoblasts. Therefore, in certain embodiments, the compositions of the invention are for use in preventing bone erosion or cartilage damage in the treatment of RA. In certain embodiments, the compositions are for use in treating patients that exhibit bone erosion or cartilage damage or patients identified as at risk of bone erosion or cartilage damage.

TNF-α is also associated with RA, but TNF-α is not involved in the pathogenesis of the later stages of the disease. In contrast, IL-17 has a role throughout all stages of chronic disease [50]. Therefore, in certain embodiments the compositions of the invention are for use in treating chronic RA or late-stage RA, such as disease that includes joint destruction and loss of cartilage. In certain embodiments, the compositions of the invention are for treating patients that have previously received anti-TNF-α therapy. In certain embodiments, the patients to be treated do not respond or no longer respond to anti-TNF-α therapy.

The compositions of the invention may be useful for modulating a patient's immune system, so in certain embodiments the compositions of the invention are for use in preventing RA in a patient that has been identified as at risk of RA, or that has been diagnosed with early-stage RA. The compositions of the invention may be useful for preventing the development of RA.

The compositions of the invention may be useful for managing or alleviating RA. The compositions of the invention may be particularly useful for reducing symptoms associated with joint swelling or bone destruction. Treatment or prevention of RA may refer to, for example, an alleviation of the severity of symptoms or a reduction in the frequency of exacerbations or the range of triggers that are a problem for the patient.

### Multiple Sclerosis

In preferred embodiments, the compositions of the invention are for use in treating or preventing multiple sclerosis. Multiple sclerosis is an inflammatory disorder associated with damage to the myelin sheaths of neurons, particularly in the brain and spinal column. Multiple sclerosis is a chronic disease, which is progressively incapacitating and which evolves in episodes. IL-17 and Th17 cells may have a key role in multiple sclerosis, for example because IL-17 levels may correlate with multiple sclerosis lesions, IL-17 can disrupt blood brain barrier endothelial cell tight junctions, and Th17 cells can migrate into the central nervous system and cause neuronal loss [51]. MicroRNA-mediated knockdown of miR-155 expression results in reduced numbers of Th1 and Th17 cells and milder symptoms in a mouse model of multiple sclerosis [52]. Therefore, the compositions of the invention may be particularly effective for preventing or treating multiple sclerosis.

In certain embodiments, treatment with the compositions of the invention results in a reduction in disease incidence or disease severity. In certain embodiments, the compositions of the invention are for use in reducing disease incidence or disease severity. In certain embodiments, treatment with the compositions of the invention prevents a decline in motor function or results in improved motor function. In certain embodiments, the compositions of the invention are for use in preventing a decline in motor function or for use in improving motor function. In certain embodiments, treatment with the compositions of the invention prevents the development of paralysis. In certain embodiments, the compositions of the invention are for use in preventing paralysis in the treatment of multiple sclerosis.

The compositions of the invention may be useful for modulating a patient's immune system, so in certain embodiments the compositions of the invention are for use in preventing multiple sclerosis in a patient that has been identified as at risk of multiple sclerosis, or that has been diagnosed with early-stage multiple sclerosis or “relapsing-remitting” multiple sclerosis. The compositions of the invention may be useful for preventing the development of sclerosis.

The compositions of the invention may be useful for managing or alleviating multiple sclerosis. The compositions of the invention may be particularly useful for reducing symptoms associated with multiple sclerosis. Treatment or prevention of multiple sclerosis may refer to, for example, an alleviation of the severity of symptoms or a reduction in the frequency of exacerbations or the range of triggers that are a problem for the patient.

In alternative embodiments, the compositions of the invention are for use in treating or preventing a disease that is not multiple sclerosis. In certain embodiments, the compositions of the invention are for use in treating or preventing a disease that is not associated with the nervous system. In certain embodiments, the compositions of the invention are for use in treating or preventing a disease that is distal to the GI tract and is not associated with the nervous system.

### Psoriasis

Psoriasis is a chronic inflammatory skin disease. Th1 and Th17 levels are higher in psoriasis patients compared with healthy control subjects. The clinical improvement following treatment of psoriasis patients with the anti-TNF-α antagonist adalimumab is associated with a decline in the frequency of Th1 and Th17 cells and their associated cytokines [53]. GVHD can also affect the skin (e.g. rashes). Therefore, the compositions of the invention may be useful for treating or preventing psoriasis in a subject.

In preferred embodiments, the compositions of the invention are for use in treating or preventing psoriasis. In certain embodiments, the compositions of the invention are for use in treating or preventing psoriasis, wherein said treatment or prevention is achieved by reducing or preventing elevation of the Th17 and/or Th1 inflammatory response.

### Systemic Lupus Erythematosus

Systemic lupus erythematosus (SLE) is an autoimmune disease. SLE patients have been found to have significantly higher levels of Th17 cytokines and an altered balance of Th1 and Th17 cell responses relative to healthy control subjects [54,55]. Therefore, the compositions of the invention may be useful for treating or preventing systemic lupus erythematosus in a subject.

In preferred embodiments, the compositions of the invention are for use in treating or preventing SLE. In certain embodiments, the compositions of the invention are for use in treating or preventing SLE, wherein said treatment or prevention is achieved by reducing or preventing elevation of the Th17 and/or Th1 inflammatory response.

### Allograft Rejection

Allograft rejection occurs when transplanted tissues are rejected by the recipient's immune system. A number of clinical studies have shown that serum and intragraft levels of IFN-γ and IL-17 positively correlate with acute rejection [56]. Allograft rejection and GVHD both follow transplants and are related to alloimmunity. Therefore, the compositions of the invention may be useful for treating or preventing allograft rejection in a subject.

In preferred embodiments, the compositions of the invention are for use in treating or preventing allograft rejection. In certain embodiments, the compositions of the invention are for use in treating or preventing allograft rejection, wherein said treatment or prevention is achieved by reducing or preventing elevation of the Th17 and/or Th1 inflammatory response.

In certain embodiments, the compositions of the invention may be administered after the patient has received the transplant. In certain embodiments, the compositions of the invention may be administered before the transplant. Administration of the compositions of the invention before the transplant has been received may be useful in priming the immune system of the subject to not elicit an inflammatory or autoimmune response against the transplanted tissue. In certain embodiments, the compositions of the invention may be used for preventing the onset of allograft rejection. In certain embodiments, the composition of the invention may be for use in the treatment or prevention of allograft rejection prophylactically. In certain embodiments, the compositions of the invention may be for use in a method of preventing transplant tissue rejection in a subject.

### Modes of Administration

Preferably, the compositions of the invention are to be administered to the gastrointestinal tract in order to enable delivery to and/or partial or total colonisation of the intestine with the bacterial strain of the invention. Generally, the compositions of the invention are administered orally, but they may be administered rectally, intranasally, or via buccal or sublingual routes.

In certain embodiments, the compositions of the invention are to be administered to a patient that has not received, for example in the preceding day, week or month or at all, an antibiotic with high activity against anaerobes, such as metronidazole, pipera-cillin-tazobactam (pip-taxo or P/T) or imipenem. In certain embodiments, the compositions of the invention are to be administered to a patient that has not received any antibiotic, such as has not received any antibiotic in the preceding day, week or month. In certain embodiments, the compositions of the invention are to be administered as a monotherapy. In certain embodiments, the compositions of the invention are to be administered alone and not in combination with any other therapeutics, such as antibiotics. In certain embodiments, the compositions of the invention are to be administered not in combination with an antibiotic having high activity against anaerobes, such as metronidazole, pipera-cillin-tazobactam (pip-taxo or P/T) or imipenem. The examples demonstrate that the compositions of the invention are effective without requiring clearance of the gut microbiome with antibiotics. In certain embodiments, the compositions of the invention are for administration to a patient with a normal gut microbiome. In certain embodiments, the compositions of the invention are for administration to a patient that has normal levels, increased levels, or does not have reduced levels of Clostridiales, Blautia, B. coccoides or B. producta. In certain embodiments, the compositions of the invention are for administration to a patient that has measurable gut colonisation of B. producta and/or B. coccoides. The examples demonstrate that the compositions of the invention do not require any particular gut microbiome condition to be effective at treating disease.

In certain embodiments, the compositions of the invention may be administered as a foam, as a spray or a gel.

In certain embodiments, the compositions of the invention may be administered as a suppository, such as a rectal suppository, for example in the form of a theobroma oil (cocoa butter), synthetic hard fat (e.g. suppocire, witepsol), glycero-gelatin, polyethylene glycol, or soap glycerin composition.

In certain embodiments, the composition of the invention is administered to the gastrointestinal tract via a tube, such as a nasogastric tube, orogastric tube, gastric tube, jejunostomy tube (J tube), percutaneous endoscopic gastrostomy (PEG), or a port, such as a chest wall port that provides access to the stomach, jejunum and other suitable access ports.

The compositions of the invention may be administered once, or they may be administered sequentially as part of a treatment regimen. In certain embodiments, the compositions of the invention are to be administered daily.

In certain embodiments of the invention, treatment according to the invention is accompanied by assessment of the patient's gut microbiota. Treatment may be repeated if delivery of and/or partial or total colonisation with the strain of the invention is not achieved such that efficacy is not observed, or treatment may be ceased if delivery and/or partial or total colonisation is successful and efficacy is observed.

In certain embodiments, the composition of the invention may be administered to a pregnant animal, for example a mammal such as a human in order to prevent an inflammatory or autoimmune disease developing in her child in utero and/or after it is born.

The compositions of the invention may be administered to a patient that has been diagnosed with GVHD or an inflammatory or autoimmune disease, or that has been identified as being at risk of GVHD or an inflammatory or autoimmune disease. The compositions may also be administered as a prophylactic measure to prevent the development of GVHD or an inflammatory or autoimmune disease in a healthy patient.

The compositions of the invention may be administered to a patient that has been identified as having an abnormal gut microbiota. For example, the patient may have reduced or absent colonisation by Blautia, and in particular Blautia producta and/or Blautia coccoides.

The compositions of the invention may be administered as a food product, such as a nutritional supplement.

Generally, the compositions of the invention are for the treatment of humans, although they may be used to treat animals including monogastric mammals such as poultry, pigs, cats, dogs, horses or rabbits. The compositions of the invention may be useful for enhancing the growth and performance of animals. If administered to animals, oral gavage may be used.

### Compositions

Generally, the composition of the invention comprises bacteria. In preferred embodiments of the invention, the composition is formulated in freeze-dried form. For example, the composition of the invention may comprise granules or gelatin capsules, for example hard gelatin capsules, comprising a bacterial strain of the invention.

Preferably, the composition of the invention comprises lyophilised bacteria. Lyophilisation of bacteria is a well-established procedure and relevant guidance is available in, for example, references [57-59].

Alternatively, the composition of the invention may comprise a live, active bacterial culture.

In preferred embodiments, the composition of the invention is encapsulated to enable delivery of the bacterial strain to the intestine. Encapsulation protects the composition from degradation until delivery at the target location through, for example, rupturing with chemical or physical stimuli such as pressure, enzymatic activity, or physical disintegration, which may be triggered by changes in pH. Any appropriate encapsulation method may be used. Exemplary encapsulation techniques include entrapment within a porous matrix, attachment or adsorption on solid carrier surfaces, self-aggregation by flocculation or with cross-linking agents, and mechanical containment behind a microporous membrane or a microcapsule. Guidance on encapsulation that may be useful for preparing compositions of the invention is available in, for example, references [60] and [61].

The composition may be administered orally and may be in the form of a tablet, capsule or powder. Encapsulated products are preferred because Blautia are anaerobes. Other ingredients (such as vitamin C, for example), may be included as oxygen scavengers and prebiotic substrates to improve the delivery and/or partial or total colonisation and survival in vivo. Alternatively, the probiotic composition of the invention may be administered orally as a food or nutritional product, such as milk or whey based fermented dairy product, or as a pharmaceutical product.

The composition may be formulated as a probiotic.

A composition of the invention includes a therapeutically effective amount of a bacterial strain of the invention. A therapeutically effective amount of a bacterial strain is sufficient to exert a beneficial effect upon a patient. A therapeutically effective amount of a bacterial strain may be sufficient to result in delivery to and/or partial or total colonisation of the patient's intestine.

A suitable daily dose of the bacteria, for example for an adult human, may be from about 1×10³ to about 1×10¹¹ colony forming units (CFU); for example, from about 1×10⁷ to about 1×10¹⁰ CFU; in another example from about 1×10⁶ to about 1×10¹⁰ CFU; in another example from about 1×10⁷ to about 1×10¹¹ CFU; in another example from about 1×10⁸ to about 1×10¹⁰ CFU; in another example from about 1×10⁸ to about 1×10¹¹ CFU.

In certain embodiments, the dose of the bacteria is at least 10⁹ cells per day, such as at least 10¹⁰, at least 10¹¹, or at least 10¹² cells per day.

In certain embodiments, the composition contains the bacterial strain in an amount of from about 1×10⁶ to about 1×10¹¹ CFU/g, respect to the weight of the composition; for example, from about 1×10⁸ to about 1×10¹⁰ CFU/g. The dose may be, for example, 1 g, 3 g, 5 g, and 10 g.

In certain embodiments, the invention provides the above pharmaceutical composition, wherein the amount of the bacterial strain is from about 1×10³ to about 1×10¹¹ colony forming units per gram with respect to a weight of the composition.

In certain embodiments, the pharmaceutical composition comprises 16, 15, 14, 13, 12, 11, 10, 9, 8, 7, 6, 5 or fewer distinct bacterial species. In certain embodiments, the pharmaceutical composition comprises 4 or fewer distinct bacterial species. In certain embodiments, the pharmaceutical composition comprises 3 or fewer distinct bacterial species. In certain embodiments, the pharmaceutical composition comprises 2 or fewer distinct bacterial species. In certain embodiments, the pharmaceutical composition comprises Blautia producta or Blautia coccoides and no other bacterial species. In preferred embodiments, the compositions of the invention comprise a single strain of Blautia producta or Blautia coccoides and no other bacterial strains or species. Such compositions may comprise only de minimis or biologically irrelevant amounts of other bacterial strains or species. Strikingly, the examples demonstrate that compositions comprising only a single strain of the invention can have a potent effect on disease, with no reliance on co-administration with other strains or species.

In certain embodiments, the invention provides the above pharmaceutical composition, wherein the composition is administered at a dose of between 500 mg and 1000 mg, between 600 mg and 900 mg, between 700 mg and 800 mg, between 500 mg and 750 mg or between 750 mg and 1000 mg. In certain embodiments, the invention provides the above pharmaceutical composition, wherein the lyophilised bacteria in the pharmaceutical composition is administered at a dose of between 500 mg and 1000 mg, between 600 mg and 900 mg, between 700 mg and 800 mg, between 500 mg and 750 mg or between 750 mg and 1000 mg.

In embodiments, a probiotic, such as the composition of the invention, is optionally combined with at least one suitable prebiotic compound. A prebiotic compound is usually a non-digestible carbohydrate such as an oligo- or polysaccharide, or a sugar alcohol, which is not degraded or absorbed in the upper digestive tract. Known prebiotics include commercial products such as inulin and transgalacto-oligosaccharides. In alternative embodiments the composition of the invention does not comprise and/or is not administered in combination with a prebiotic.

In certain embodiments, the probiotic composition of the present invention includes a prebiotic compound in an amount of from about 1 to about 30% by weight, respect to the total weight composition, (e.g. from 5 to 20% by weight). Carbohydrates may be selected from the group consisting of: fructo-oligosaccharides (or FOS), short-chain fructo-oligosaccharides, inulin, isomalt-oligosaccharides, pectins, xylo-oligosaccharides (or XOS), chitosan-oligosaccharides (or COS), beta-glucans, arable gum modified and resistant starches, polydextrose, D-tagatose, acacia fibers, carob, oats, and citrus fibers. In one aspect, the prebiotics are the short-chain fructo-oligosaccharides (for simplicity shown herein below as FOSs-c.c); said FOSs-c.c. are not digestible carbohydrates, generally obtained by the conversion of the beet sugar and including a saccharose molecule to which three glucose molecules are bonded.

The compositions of the invention may comprise pharmaceutically acceptable excipients or carriers. Examples of such suitable excipients may be found in the reference [62]. Acceptable carriers or diluents for therapeutic use are well known in the pharmaceutical art and are described, for example, in reference [63]. Examples of suitable carriers include lactose, starch, glucose, methyl cellulose, magnesium stearate, mannitol, sorbitol and the like. Examples of suitable diluents include ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilizers, dyes and even flavouring agents may be provided in the pharmaceutical composition. Examples of preservatives include sodium benzoate, sorbic acid and esters of p-hydroxybenzoic acid. Antioxidants and suspending agents may be also used.

The compositions of the invention may be formulated as a food product. For example, a food product may provide nutritional benefit in addition to the therapeutic effect of the invention, such as in a nutritional supplement. Similarly, a food product may be formulated to enhance the taste of the composition of the invention or to make the composition more attractive to consume by being more similar to a common food item, rather than to a pharmaceutical composition. In certain embodiments, the composition of the invention is formulated as a milk-based product. The term “milk-based product” means any liquid or semi-solid milk- or whey-based product having a varying fat content. The milk-based product can be, e.g., cow's milk, goat's milk, sheep's milk, skimmed milk, whole milk, milk recombined from powdered milk and whey without any processing, or a processed product, such as yoghurt, curdled milk, curd, sour milk, sour whole milk, butter milk and other sour milk products. Another important group includes milk beverages, such as whey beverages, fermented milks, condensed milks, infant or baby milks; flavoured milks, ice cream; milk-containing food such as sweets.

In certain embodiments, the compositions of the invention contain a single bacterial strain or species and do not contain any other bacterial strains or species. Such compositions may comprise only de minimis or biologically irrelevant amounts of other bacterial strains or species. Such compositions may be a culture that is substantially free from other species of organism. In certain embodiments, the compositions of the invention consist of 1, 2, 3, 4, 5, 6, 7, 8, 9, 10, 11, 12, 13, 14, 15 or 16 bacterial strains or species. In certain embodiments, the compositions consist of from 1 to 10, preferably from 1 to 5 bacterial strains or species.

In certain embodiments, the compositions of the invention do not comprise any bacterial strains or species that produce butyrate.

The compositions for use in accordance with the invention may or may not require marketing approval.

In some cases, the lyophilised bacterial strain is reconstituted prior to administration. In some cases, the reconstitution is by use of a diluent described herein.

The compositions of the invention can comprise pharmaceutically acceptable excipients, diluents or carriers.

In certain embodiments, the invention provides a pharmaceutical composition comprising: a bacterial strain of the invention; and a pharmaceutically acceptable excipient, carrier or diluent; wherein the bacterial strain is in an amount sufficient to treat a disorder when administered to a subject in need thereof; and wherein the disorder is selected from the group consisting of GVHD and inflammatory or autoimmune diseases.

In certain embodiments, the invention provides pharmaceutical composition comprising: a bacterial strain of the invention; and a pharmaceutically acceptable excipient, carrier or diluent; wherein the bacterial strain is in an amount sufficient to treat or prevent GVHD, or inflammatory or autoimmune diseases.

In certain embodiments, the invention provides the above pharmaceutical composition, wherein the amount of the bacterial strain is from about 1×10³ to about 1×10¹¹ colony forming units per gram with respect to a weight of the composition.

In certain embodiments, the invention provides the above pharmaceutical composition, wherein the composition is administered at a dose of 1 g, 3 g, 5 g or 10 g.

In certain embodiments, the invention provides the above pharmaceutical composition, wherein the composition is administered by a method selected from the group consisting of oral, rectal, subcutaneous, nasal, buccal, and sublingual.

In certain embodiments, the invention provides the above pharmaceutical composition, comprising a carrier selected from the group consisting of lactose, starch, glucose, methyl cellulose, magnesium stearate, mannitol and sorbitol.

In certain embodiments, the invention provides the above pharmaceutical composition, comprising a diluent selected from the group consisting of ethanol, glycerol and water.

In certain embodiments, the invention provides the above pharmaceutical composition, comprising an excipient selected from the group consisting of starch, gelatin, glucose, anhydrous lactose, free-flow lactose, beta-lactose, corn sweetener, acacia, tragacanth, sodium alginate, carboxymethyl cellulose, polyethylene glycol, sodium oleate, sodium stearate, magnesium stearate, sodium benzoate, sodium acetate and sodium chloride.

In certain embodiments, the invention provides the above pharmaceutical composition, further comprising at least one of a preservative, an antioxidant and a stabilizer.

In certain embodiments, the invention provides the above pharmaceutical composition, comprising a preservative selected from the group consisting of sodium benzoate, sorbic acid and esters of p-hydroxybenzoic acid.

In certain embodiments, the invention provides the above pharmaceutical composition, wherein said bacterial strain is lyophilised.

In certain embodiments, the invention provides the above pharmaceutical composition, wherein when the composition is stored in a sealed container at about 4° C. or about 25° C. and the container is placed in an atmosphere having 50% relative humidity, at least 80% of the bacterial strain as measured in colony forming units, remains after a period of at least about: 1 month, 3 months, 6 months, 1 year, 1.5 years, 2 years, 2.5 years or 3 years.

### Culturing Methods

The bacterial strains for use in the present invention can be cultured using standard microbiology techniques as detailed in, for example, references [64-66].

The solid or liquid medium used for culture may be YCFA agar or YCFA medium. YCFA medium may include (per 100 ml, approximate values): Casitone (1.0 g), yeast extract (0.25 g), NaHCO₃ (0.4 g), cysteine (0.1 g), K₂HPO₄ (0.045 g), KH₂PO₄ (0.045 g), NaCl (0.09 g), (NH₄)₂SO₄ (0.09 g), MgSO₄.7H₂O (0.009 g), CaCl₂) (0.009 g), resazurin (0.1 mg), hemin (1 mg), biotin (1 μg), cobalamin (1 μg), p-aminobenzoic acid (3 μg), folic acid (5 μg), and pyridoxamine (15 μg).

### Bacterial Strains for Use in Vaccine Compositions

The inventors have identified that the bacterial strains of the invention are useful for treating or preventing GVHD. This is likely to be a result of the effect that the bacterial strains of the invention have on the host immune system. Therefore, the compositions of the invention may also be useful for preventing GVHD, and other inflammatory and autoimmune diseases, when administered as vaccine compositions. In certain such embodiments, the bacterial strains of the invention may be killed, inactivated or attenuated. In certain such embodiments, the compositions may comprise a vaccine adjuvant. In certain embodiments, the compositions are for administration via injection, such as via subcutaneous injection.

### General

The practice of the present invention will employ, unless otherwise indicated, conventional methods of chemistry, biochemistry, molecular biology, immunology and pharmacology, within the skill of the art. Such techniques are explained fully in the literature. See, e.g., references [67] and [68,74], etc.

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

The word “substantially” does not exclude “completely” e.g. a composition which is “substantially free” from Y may be completely free from Y. Where necessary, the word “substantially” may be omitted from the definition of the invention.

References to a percentage sequence identity between two nucleotide sequences means that, when aligned, that percentage of nucleotides are the same in comparing the two sequences. This alignment and the percent homology or sequence identity can be determined using software programs known in the art, for example those described in section 7.7.18 of ref. [75]. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in ref. [76].

Unless specifically stated, a process or method comprising numerous steps may comprise additional steps at the beginning or end of the method, or may comprise additional intervening steps. Also, steps may be combined, omitted or performed in an alternative order, if appropriate.

Various embodiments of the invention are described herein. It will be appreciated that the features specified in each embodiment may be combined with other specified features, to provide further embodiments. In particular, embodiments highlighted herein as being suitable, typical or preferred may be combined with each other (except when they are mutually exclusive).

### MODES FOR CARRYING OUT THE INVENTION

### Example 1—Efficacy of Strain NCIMB 43170 in Enhancing Survival from GVHD

### Objective

The inventors sought to determine the effect of strain NCIMB 43170 on graft versus host disease (GVHD) induced in Balb/C mice.

### Material and Methods

Animals

Male Balb/C mice (BALB/cAnNCrl; 6-8 weeks old; n=125) with an average starting body weight (±SEM) of 20.67±0.11 g were obtained from Charles River Laboratories (Wilmington, Mass.). An additional n=75 male C57Bl/6 (C57Bl/6NCrl; 6-8 weeks old) were obtained from the same vendor. Animals were acclimatized prior to study commencement. During this period, the animals were observed daily in order to reject any that presented in poor condition.

Housing

The study was performed in animal rooms provided with HEPA filtered air at a temperature of 70±5° F. and 50%±20% relative humidity. Animals were housed in groups of 4-6 per cage. Specifically, groups with 8 animals/group were housed at n=4/cage; groups with 10 animals/group were housed at n=5/cage; and groups with 12 animals per group were housed at n=6/cage. Animals were housed in HEPA-filtered, individually ventilated cages. Cages were geographically separated on the racks to minimize cross-contamination between groups. Animal rooms were set to maintain a minimum of 12 to 15 air changes per hour. The room was on an automatic timer for a light/dark cycle of 12 hours on and 12 hours off with no twilight. Alpha-Dri® bedding (irradiated) was used. In addition to bedding, each cage was provided with enviro-dri and a shepherd shack (enrichment). Floors were swept daily and mopped a minimum of twice weekly with a commercial detergent. Walls and cage racks were sponged a minimum of once per month with a dilute bleach solution. A cage card or label with the appropriate information necessary to identify the study, dose, animal number, and treatment group was used to mark all cages. The temperature and relative humidity was recorded during the study, and the records retained. All technicians donned PPE (lab coat, gloves, safety goggles) prior to entering the lab/vivarium and working with animals.

Diet

Animals were fed with LabDiet 5053 sterile (irradiated) rodent chow and water (reverse osmosis) was provided ad libitum. No food-based enrichment was provided.

Animal Randomization and Allocations

Animals were randomized into 6 groups at the start of the study. Each group comprised between 8 and 12 mice. Each group was further sub-divided into cohorts A and B (n=4-6 mice per group per cohort); cohorts had staggered disease timelines.

Analysis of Growth Kinetics of Strain NCIMB 43170

Prior to administration of strain NCIMB 43170 growth curve/maximum OD were determined and virtual colony count (VCC) at maximum OD600 and after wash were determined. Growth curve/maximum OD analysis took place as follows. At 6 AM, one tube each of frozen bacterial stocks were brought into the Coy chamber. Tubes were allowed to thaw, were mixed carefully by pipetting up and down, and two tubes (duplicates) containing 9.5 mL of pre-reduced, pre-warmed (37° C.) YCFA broth were inoculated with 500 μL bacterial stock. These were the pre-cultures. Pre-cultures were incubated at 37° C. in the Coy chamber for 24 hours. At 6 AM the next day (i.e. after 24 hours of incubation), a small aliquot of each culture was removed from the Coy chamber, and the OD600 was determined by nanodrop. Tubes were mixed by inversion prior to removing the aliquot for OD600 measurement. The remainder of the 24 hour cultures (using the tube with the higher OD600 as determined above) were cultured in duplicate as follows: 250 μL of strain NCIMB 43170 24 hour culture was used to inoculate two tubes containing 24.75 mL pre-warmed YCFA broth. These cultures were incubated at 37° C. in the Coy chamber for 24 hours, and a small aliquot was removed from the Coy chamber for measurement of OD600 every two hours for 16 hours (i.e. at 8 AM, 10 AM, 12 PM, 2 PM, 4 PM, 6 PM, 8 PM, and 10 PM), and at 24 hours (6 AM the next day). Tubes were mixed by inversion prior to removing the aliquot for OD600 measurement.

VCC at maximum OD analysis occurred as follows: one tube of strain NCIMB 43170 stock was brought into the Coy chamber. Tubes were allowed to thaw, were mixed carefully by pipetting up and down, and two tubes (duplicates) containing 9.5 mL of pre-reduced, pre-warmed YCFA broth was inoculated with 500 μL bacterial stock. These were the pre-cultures. Pre-cultures were incubated at 37° C. in the Coy chamber for 24 hours. The next day (after 24 hours of incubation), a small aliquot the pre-culture was removed from the Coy chamber, and the OD600 was determined by nanodrop. Tubes were mixed by inversion prior to removing the aliquot for OD600 measurement. The remainder of the 24 hour, cultures (using the tubes with the higher OD) were cultured in duplicate as follows: 250 μL was used to inoculate two tubes containing 24.75 mL pre-warmed YCFA broth. These were the main cultures. A small aliquot of main culture was removed from the Coy chamber at the indicated time, and the OD600 was determined by nanodrop. Tubes were mixed by inversion prior to removing the aliquot for OD600 measurement. The VCC of the remaining stock was determined as follows: an individual dilution series (undiluted, 1:10³, 1:10⁴, 1:10⁵, and 1:10⁶) was prepared in PBS. The remainder of each culture was then transferred to a 50 mL conical tube, and the tubes were removed from the Coy chamber and centrifuged (3500×g; 15 minutes). Once centrifugation was complete, the tubes were returned to the Coy chamber, and the supernatants were removed (with care taken to avoid disturbing the pellets), and measured. The pellets were resuspended in volumes of PBS equivalent to that of the removed supernatants, and were mixed carefully with a pipette (no vortexing). An individual dilution series (undiluted, 1:10³, 1:10⁴, 1:10⁵, and 1:10⁶) was prepared in PBS. Both dilution series (broth and PBS suspended) were spot plated (20 μL) in triplicate in one quadrant of a pre-reduced YCFA agar plate. Plates were incubated at 37° C. in the Coy chamber for 48 hours, and the VCC of whichever dilution yielded spots with 5-20 CFU/spot was counted. The three spot VCC/spot values were averaged to determine the VCC/mL of overnight cultures in broth and centrifuged/resuspended in PBS.

Strain NCIMB 43170 Dosage Preparation

Two days prior to each dosing timepoint, one tube (1 mL/tube) per strain of frozen stocks of strain NCIMB 43170 were entered into the Coy chamber. The tubes were allowed to thaw, and two 15 mL tubes, each containing 9.5 mL of pre-reduced and pre-warmed YCFA broth were inoculated with 0.5 mL of each bacteria stock. These were the pre-cultures (tubes 1 and 2). Pre-cultures were incubated at 37° C. in the Coy chamber for 24 hours.

After incubation for 24 hours, one day before each dosing timepoint, cultures were mixed by inversion, and a small aliquot (20 μL) of the cultures were removed from the Coy chamber for OD600 determination by nanodrop. Whichever tube (1 or 2) per strain had the higher OD600 value was used for the main culture, in duplicate, as follows: 2.5 mL of the pre-culture with the higher OD600 was used to inoculate 22.5 mL of pre-warmed YCFA broth in a 50 mL conical (in duplicate; tubes A and B). These main cultures were incubated in the Coy chamber (at 37°) for 24 hours.

On each dosing day (after the above main culture incubations), cultures were mixed by inversion, and a small aliquot (20 μL) of the cultures were removed from the Coy chamber for OD600 determination by nanodrop. Whichever tube (A or B) has the higher OD600 value was removed from the Coy chamber and centrifuged (3500×g; 15 minutes). Once centrifugation was complete, the tubes were returned to the Coy chamber, and the supernatant was removed (with care taken to avoid disturbing the pellet) by pipette. Pellets were resuspended in 2.49 ml PBS. Pellets were mixed carefully by pipetting (no vortexing). An aliquot (0.5 mL) of the resuspended culture was pipetted into Eppendorf tubes and retained in the Coy chamber. The remainder of the resuspended culture was removed from the Coy chamber and used for dosing (0.1 mL per animal), with care taken to dose the animals as quickly as possible following the resuspension.

The 0.5 mL aliquot of each strain retained in the Coy chamber was used for preparation of an individual dilution series in pre-reduced MRD; the 1:10⁵, 1:10⁶, 1:10⁷, and 1:10⁸ dilutions were spot plated (20 μL) in triplicate in one quadrant of a pre-reduced YCFA agar plate. Plates were incubated at 37° C. in the Coy chamber for 48 hours, and the VCC of whichever dilution yielded spots with 5-20 CFU/spot were counted. The three spot VCC/spot values were averaged to determine the VCC/mL of the experimental dosing material.

Pre-Treatment Phase

All animals were weighed and randomized by weight into treatment groups prior to study start. Prior to GVHD induction on Days −1 and 0, all animals were pre-treated (PO) with PBS (Groups 1-4), bacterial strain NCIMB 43170 (Group 8), or Butyrate salt control (Group 10) daily starting on Day −14. Butyrate was used as a positive control as butyrate deficit has been identified in the gut of GVHD patients. Treatments were administered to groups at random, and group treatments were alternated daily to prevent the same groups from being treated at the same time each day. Once test article dosing had begun, care was taken to minimize group cross-contamination: gloves were changed by the technician between treatment groups, and were sprayed with 70% isopropyl alcohol between each cage of the same group.

GVHD Modelling

GVHD was induced in n=84 Balb/C mice (Groups 4, 8 and 10) using a single acute dose of 8 Gy of total body irradiation (TBI) on Day −1. On Day 0, these recipient mice were given an intravenous injection of a combination of T cell depleted bone marrow cells and splenic cells from donor C57Bl/6 mice in PBS. Bone marrow cells were isolated using standard flushing practices, and were T cell depleted using the cell surface T cell antigen CD3, with a CD3-biotin kit (Miltenyi Biotec catalog 130-094-973). Splenocytes were isolated using Miltenyi GentleMACS Dissociators. Animals in Group 1 served as naïve controls and received neither TBI nor cell transfer. Animals in Group 2 served as irradiation controls and received the 8 Gy of TBI on Day −1, but did not receive a cell transfer on Day 0. Animals in Group 3 served as syngeneic adoptive transfer controls; these animals received 8 Gy of TBI on Day −1, and an intravenous injection of a combination of T cell depleted bone marrow cells and splenic cells from donor Balb/C mice in sterile PBS.

Daily test article dosing continued for the duration of the study (Days −14 to 30). Animal survival was recorded daily, as an indication of GVHD severity. Animals were also weighed, observed, and given a clinical GVHD score daily for the duration of the study following GVHD induction. The GVHD score was assessed by a standard scoring system based on five criteria (Table 1): percentage of weight change, posture (hunching), activity, fur texture, and skin integrity (maximum score=10). Animal handling was carried out in a pseudo-random order, alternating each day, so as to prevent the same animal from being handled at the same time each day.

TABLE 1
Assessment of GVHD in Transplanted Mice (Daily Scoring)

| Criteria | Grade 0 | Grade 1 | Grade 2 |
|---|---|---|---|
| Weight loss | <10% | >10% <25% | >25% |
| Posture | Normal | Hunching noted | Severe gait, |
| | | only at rest | impaired |
| | | | movement |
| Activity | Normal | Mild to | Stationary until |
| | | moderately | stimulated |
| | | decreased | |
| Fur texture | Normal | Mild to moderate | Severe ruffling |
| | | ruffling | and/or poor |
| | | | grooming |
| Skin integrity | Normal | Scaling of paws | Obvious areas of |
| | | and/or tail | denuded skin |

Animals that lost 20% of their body weight were administered sub-cutaneous fluids (SID; saline) and provided with softened food. If any individual study animal required softened food, all study animals were provided with softened food until that individual animal's weight loss had been rescued. Treatment continued until either scheduled euthanasia or body weight loss greater than 30%. Animals that were unable to right themselves, were cold to the touch, or were moribund were euthanized.

On Day 29, all surviving animals underwent endoscopy to monitor colonic inflammation. Images were taken and colitis severity and stool consistency were scored using the scoring scale shown in Table 2.

TABLE 2
Endoscopy Colitis Scoring Scale

| Score | Description: |
|---|---|
| 0 | Normal |
| 1 | Loss of vascularity |
| 2 | Loss of vascularity and friability |
| 3 | Friability and erosions |
| 4 | Ulcerations and bleeding |

On Day 30, blood was collected by retro-orbital bleed; blood (approximately 150-300 μL) was collected into two tubes—approximately two third of the blood was collected into K₂EDTA tubes, and the remaining one third was collected into lithium-heparin tubes. Both samples were centrifuged and processed for plasma, and plasma tubes were clearly labeled to indicate the anti-coagulant used. For the K₂EDTA sample, plasma was aliquoted as follows: 25 μL (for use in downstream citrulline assay), and remainder. All plasma was frozen at −80° C. All K₂EDTA samples were assessed for citrulline by ELISA upon study completion

Euthanasia was performed by CO₂ inhalation and cervical dislocation, without organ collections for animals euthanized off-schedule during the TBI phase of the study. Euthanasia was performed by cervical dislocation only, with organ collections, for animals euthanized off-schedule during the GVHD phase of the study. Terminal collections occurred on the benchtop. The benchtop was cleaned with 70% isopropyl alcohol and a commercial disinfectant before beginning. Instruments were cleaned with 70% isopropyl alcohol between animals, and with a commercial disinfectant between groups.

Statistical Analyses

Parametric data was analyzed by one-way ANOVA with Tukey's multiple comparisons test to compare all groups to one another. Non-parametric data was analyzed by Kruskal-Wallis test with Dunn's multiple comparisons test to compare all groups to one another. All statistical analyses were performed using GraphPad Prism 7 (La Jolla, Calif.).

### Results and Discussion

Body Weight

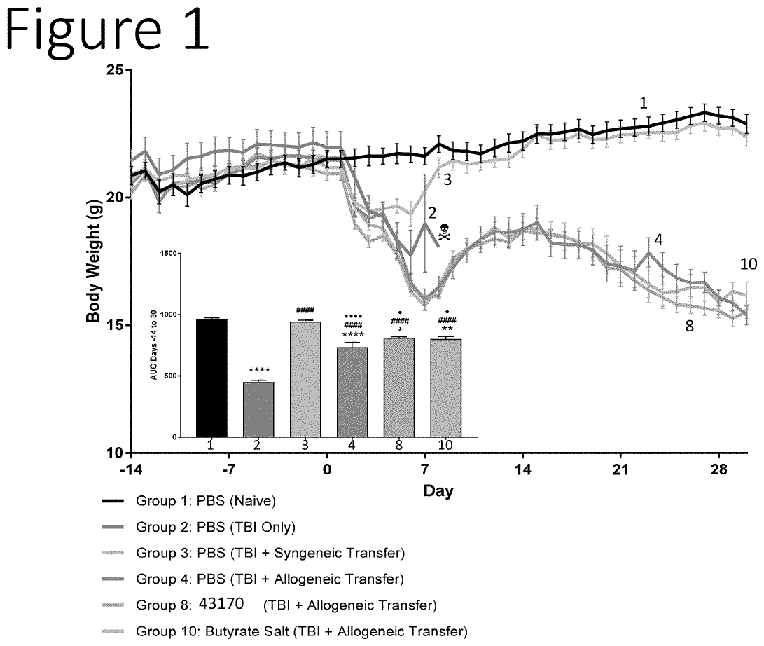

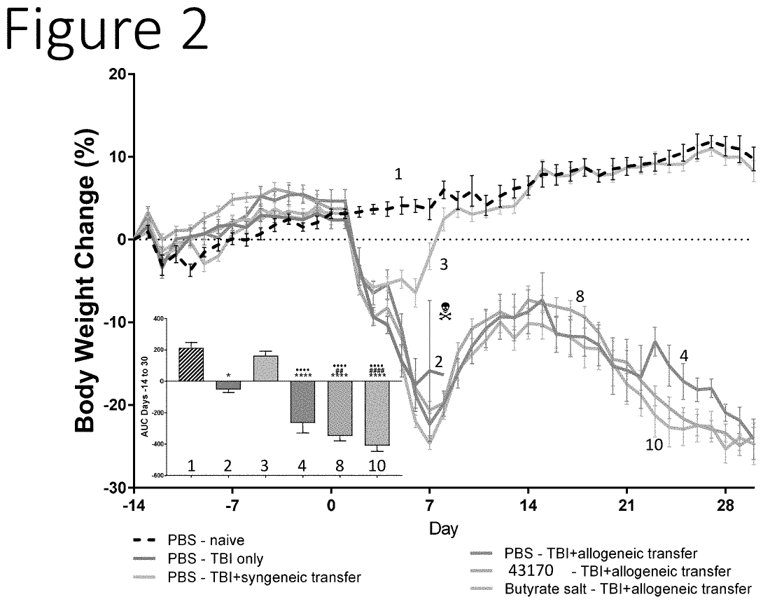

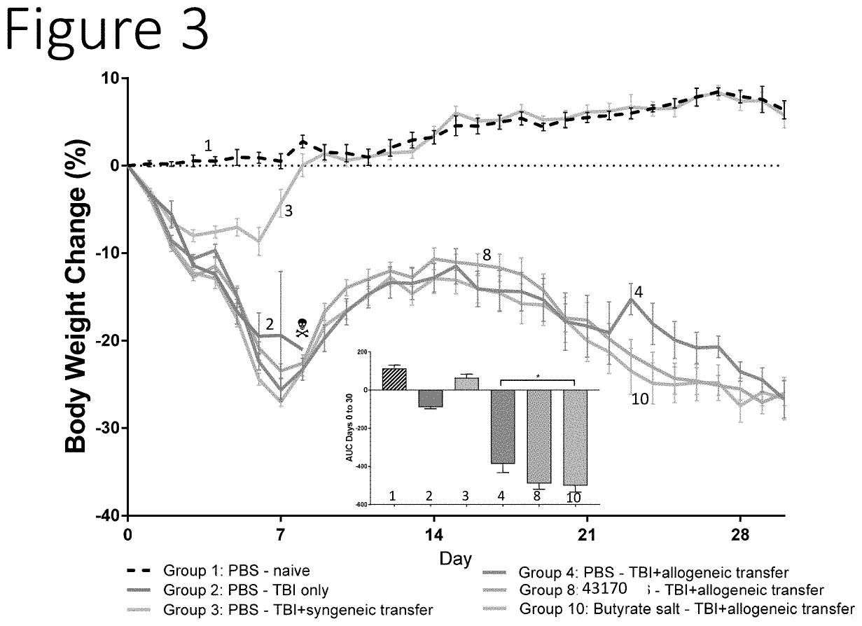

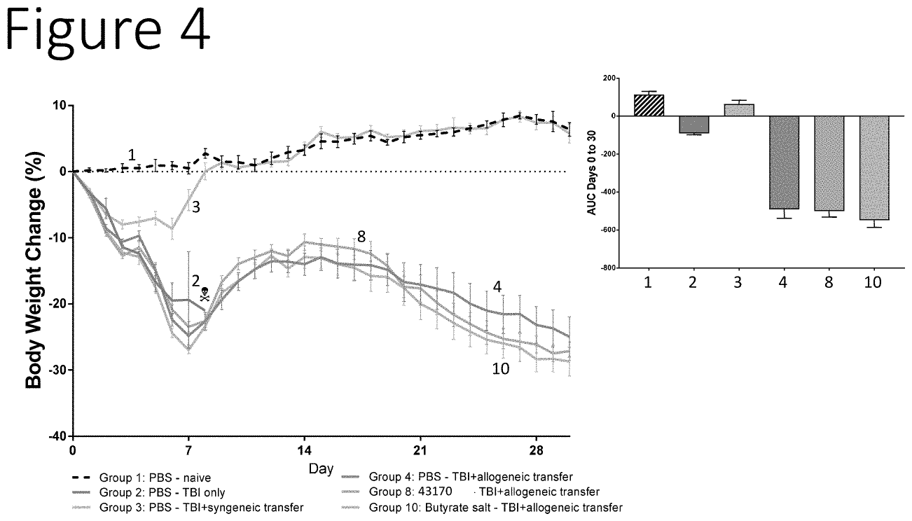

Animals were weighed on a daily basis for the duration of the study, and the mean body weight for all groups over the course of the study is shown in FIG. 1. Body weight change relative to either Day −14 (FIG. 2) or Day 0 (FIG. 3) was calculated. In order to determine statistically significant differences between groups in either mean body weight or mean body weight change relative to either Day −14 or Day 0, the area under the curve (AUC) was calculated using the trapezoidal transformation rule and is shown in the insets of FIGS. 1, 2 and 3. To account for group attrition, the body weight change relative to Day 0 shown with the body weight with which an animal died carried forward for the duration of the study for animals found dead or euthanized (for all groups with the exception of Group 2) is shown in FIG. 4, with the AUC inset.

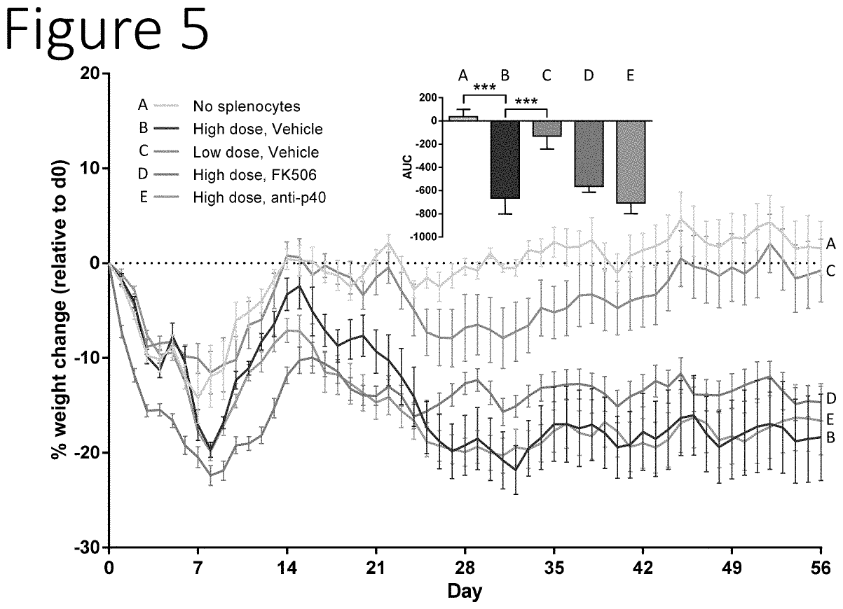

No major differences in mean body weight (FIG. 1) were observed for any groups during the pre-treatment period. All groups exposed to TBI demonstrated body weight loss from Days 0 to 3. Mean body weight for animals in Group 3 (PBS−TBI+syngeneic transfer) recovered from this point forward and ultimately returned to baseline. Mean body weight for animals in Group 2 (PBS−TBI only) failed to recover prior to the death of all animals within the group. For all other study groups, mean body weight continued to decrease through Day 7, increased through Day 14, and subsequently decreased through the duration of the study. The mean body weight in Groups 2, 8 and 10 was significantly decreased over the course of the study as compared to Group 1 (PBS—naïve). In contrast, the mean body weight in Groups 3, 4, 8 and 10 were significantly increased over the course of the study as compared to Group 2. Finally, the mean body weight in Groups 8 and 10 was significantly decreased over the course of the study as compared to Group 3. No significant differences in mean body weight over the course of the study were observed when comparing treatment groups (Groups 8 and 10) with Group 4 (PBS−TBI+allogeneic transfer). This same trend was observed when mice were administered the immunosuppressant tacrolimus, a known therapy of GVHD (FK506—FIG. 5).

The mean body weight change relative to Day −14 (FIG. 2) increased for all groups during the pre-treatment period, and the kinetics of body weight change relative to Day −14 from Day 0 onward to similar to that observed for mean body weight. Animals in Groups 2 and 4, 8 and 10 demonstrated significantly increased body weight loss over the course of the study as compared to both Group 1 and Group 3.

The mean body weight change relative to Day 0 (FIGS. 3 and 4) decreased for all groups exposed to TBI from Days 0 to 3, at which point body weight gain began for animals in Group 3; body weight loss continued for all other groups through Day 4. Body weight change relative to Day 0 increased from Day 7 to Day 14, and mean body weight loss was observed for Groups 4, 8 and 10 from Day 14 through the end of the study on Day 30. While the overall pattern in body weight change relative to Day 0 was similar regardless of whether body weight was carried forward for deceased animals, it did affect the statistical significance of certain comparisons. Significantly increased body weight loss as compared to Groups 1, 2, and 3 were observed for Groups 4, 8 and 10 both with and without body weight for deceased animals carried forward, again, which is similar to the trend observed in mice administered the known GVHD therapy tacrolimus (FK506—FIG. 5).

Survival

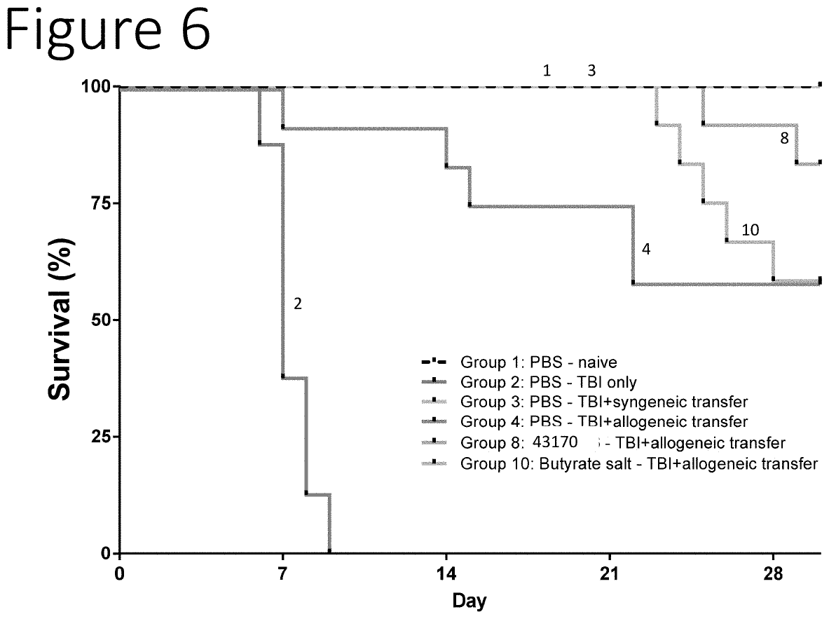

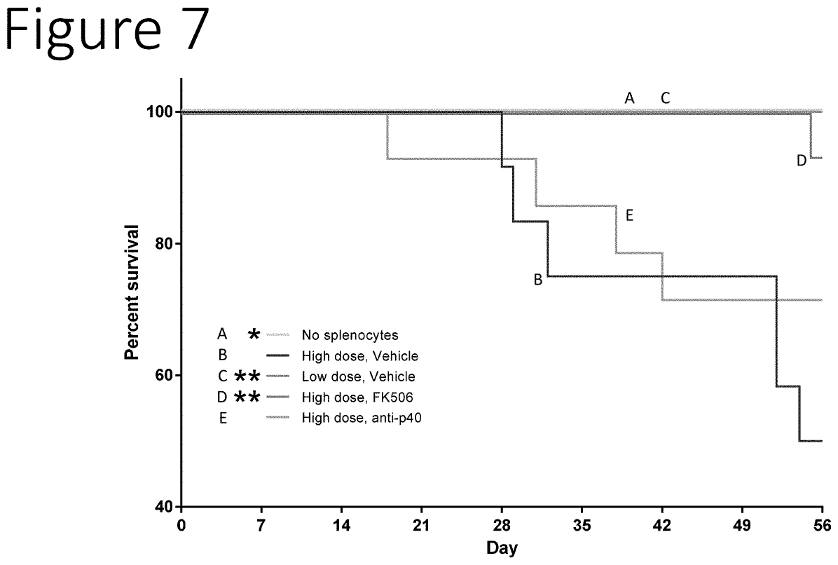

Animals were assessed daily for survival or moribundity, and a Kaplan-Meier curve showing survival over the duration of the study is shown in FIG. 6. Survival was 100% in Groups 1 and 3, 0% in Group 2, and 83.33% in Group 8. The survival observed in Group 8 was strikingly improved compared to both Groups 4 and 10. This is notable, as butyrate has been proposed as a treatment for GVHD [77]. Survival rates for mice in group 8 were comparable to mice controls administered the known GVHD treatment tacrolimus (FK506—FIG. 7). These data demonstrate that compositions comprising bacterial strains of the species Blautia producta and Blautia coccoides may be useful for treating and preventing GVHD and other inflammatory and autoimmune diseases.

GVHD Scores

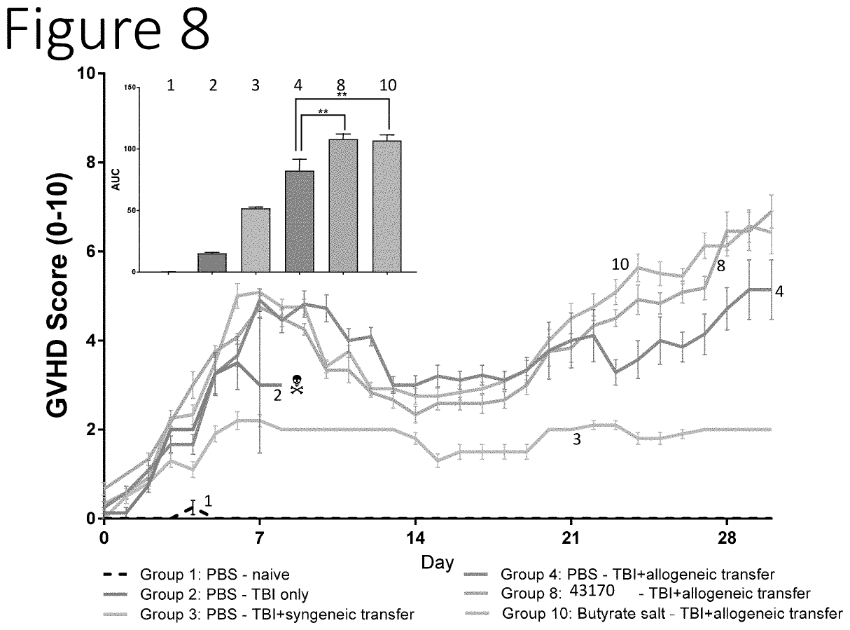

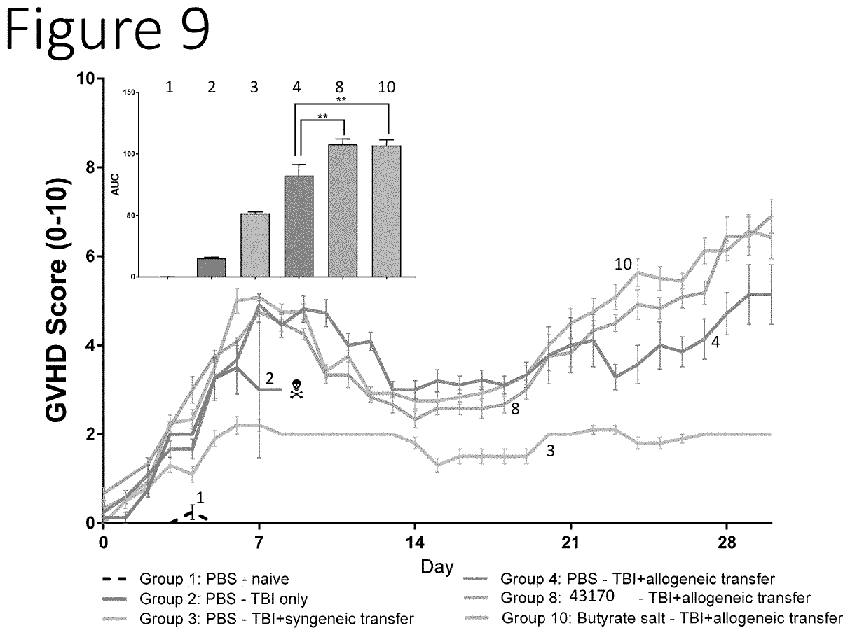

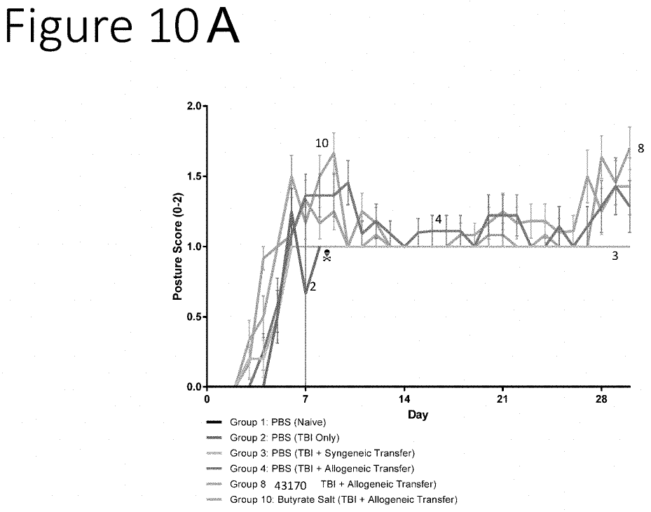

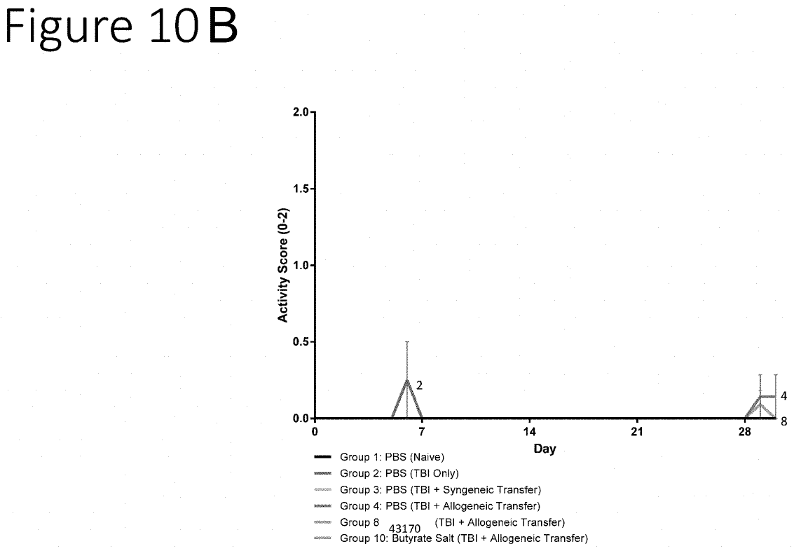

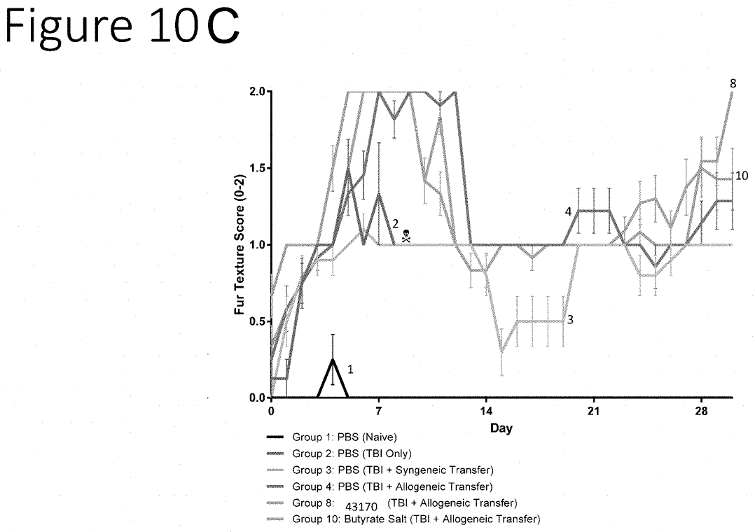

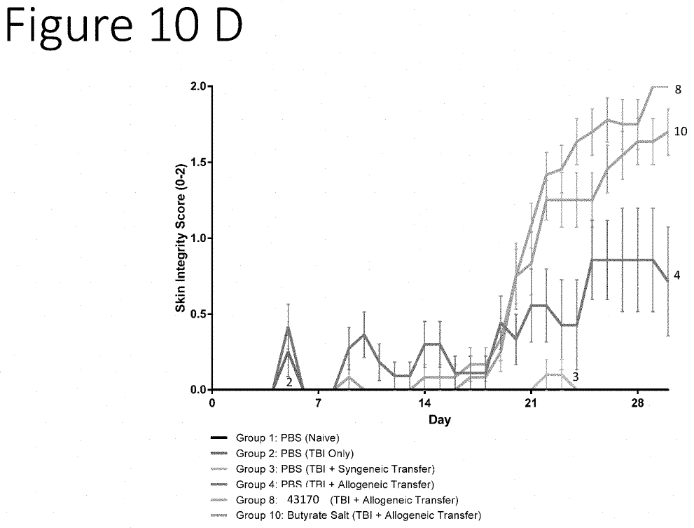

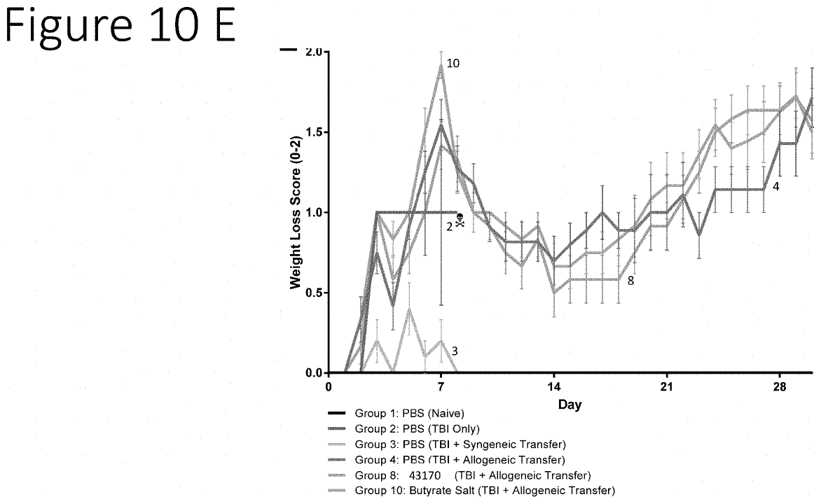

GVHD scores were assessed (as per the multi-parameter scoring shown in Table 1) in all animals from Day 0 through the end of the study on Day 30. Mean GVHD scores for all groups are shown in FIG. 8, and this same data presented with the GVHD score with which an animal died carried forward is shown in FIG. 9. The AUC was calculated using the trapezoidal transformation rule in order to determine statistically significant differences in overall GVHD scores between groups, and this is shown in the insets of FIGS. 8 and 9. The clinical GVHD score assigned to each animal is a composite consisting of posture (FIG. 10A), activity (FIG. 10B), fur texture (FIG. 10C), skin integrity (FIG. 10D) and weight loss (FIG. 10E).

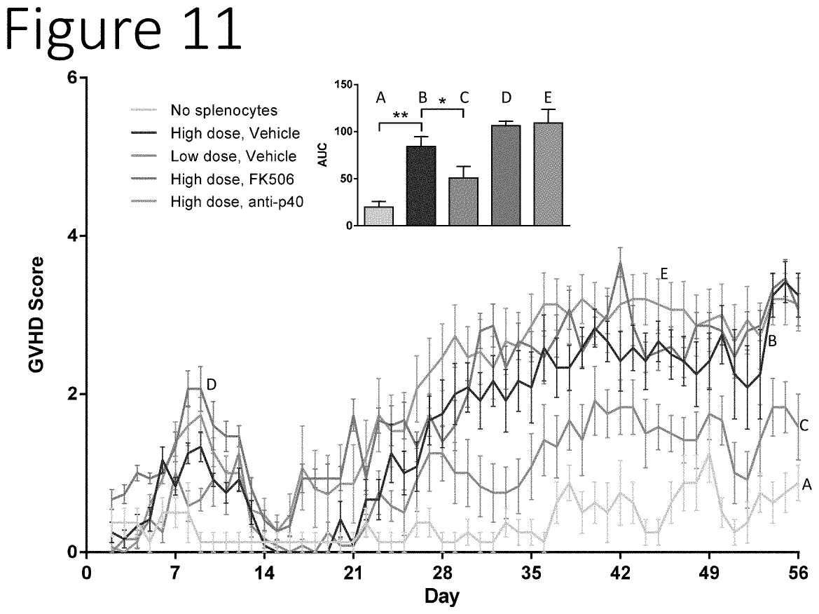

Intravenous injection of allogeneic splenocytes and bone marrow cells induced GVHD in all groups that began around Day 19 and progressively increased in severity until the conclusion of the study. There was an initial GVHD score increased between Days 0-7, presumably due to TBI and engraftment; survival of animals past this point verifies successful engraftment of the transplanted cells. While the GVHD score kinetics were similar regardless of whether GVHD score was carried forward for deceased animals, statistically significant differences between groups differed. Animals in Groups 3, 4, 8 and 10 demonstrated significantly increased mean GVHD scores as compared to both Groups 1 and 2 both with and without GVHD scores for deceased animals carried forward; likewise, animals in Groups 4, 8 and 10 demonstrated significantly increased mean GVHD scores as compared to Group 3 in both instances. This trend was also observed in mice models of GVHD administered the immunosuppressant tacrolimus, which is a known therapy of GVHD (FIG. 11).

Mice administered strain NCIMB 43170 had a similar GVHD score in comparison to mice administered butyrate salts (Group 10), which is notable considering the role of butyrate in maintaining correct barrier function. These data demonstrate that compositions comprising bacterial strains of the species Blautia producta and Blautia coccoides may be useful for treating and preventing GVHD and other inflammatory and autoimmune diseases.

Endoscopy

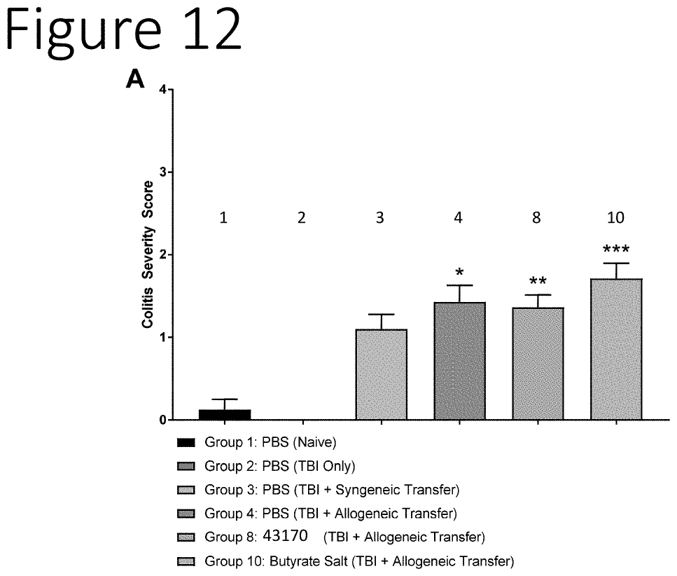

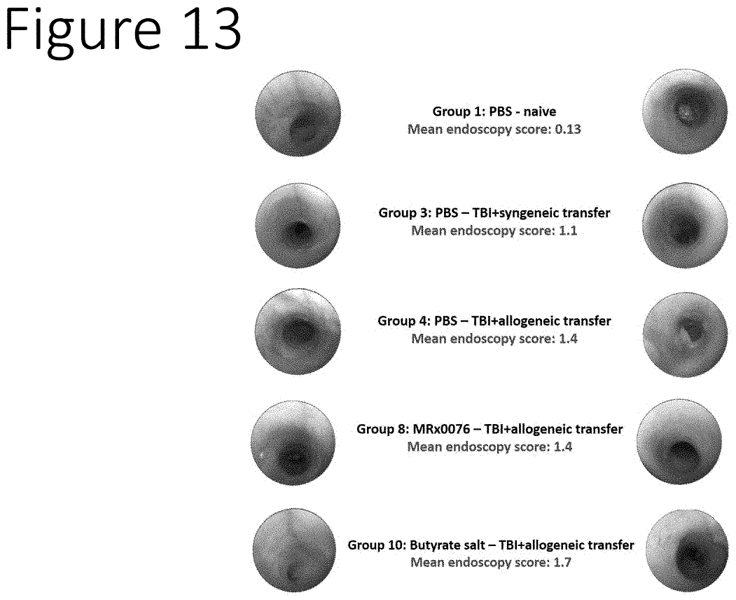

Animals underwent endoscopy on Day 29, in order to assess colonic inflammation. Colitis was scored visually on a five-point scale that ranges from 0 for normal, to 4 for severe ulceration (Table 2). The mean colitis severity is shown in FIG. 12.

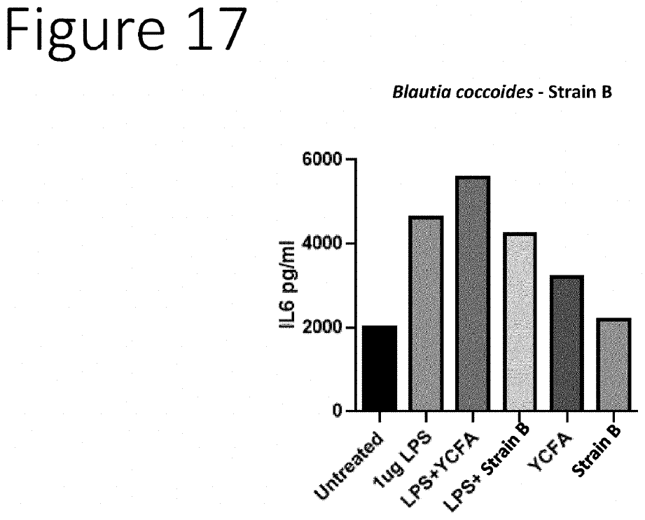

Mean colitis severity scores were increased for strain NCIMB 43170-treated and butyrate-treated animals as compared to naïve mice (Group 1). However, colitis was less severe in strain NCIMB 43170-treated mice as compared with butyrate-treated mice. This is notable, as correction of butyrate deficit has been suggested as a treatment for colitis [78]. Representative endoscopy images are shown in FIG. 17. These data demonstrate that compositions comprising bacterial strains of the species Blautia producta and Blautia coccoides may be useful for treating and preventing colitis and other inflammatory and autoimmune diseases.

Plasma Citrulline

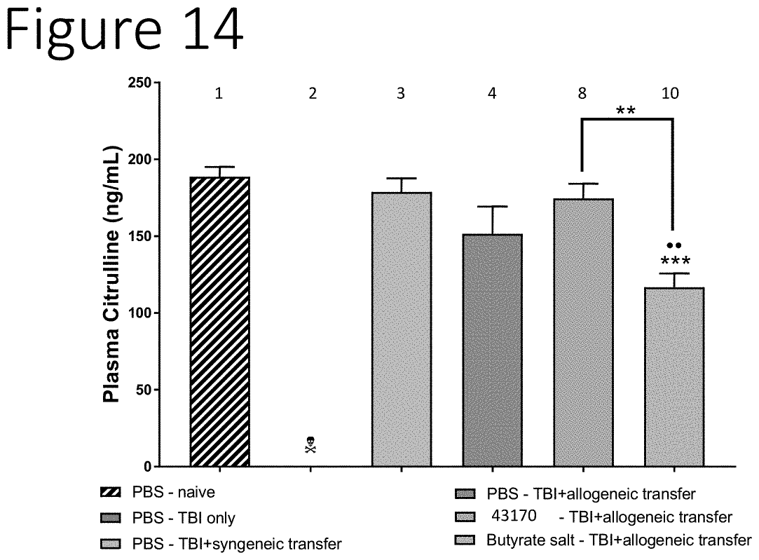

Blood was collected prior to euthanasia from all surviving animals and was processed for plasma. Plasma citrulline was assessed as a marker of intestinal permeability in duplicate by ELISA. A reduction in plasma citrulline levels corresponds to a loss in epithelial cell mass indicating an increase in gut barrier permeability. The maintenance of gut barrier function (i.e. a maintenance of gut impermeability) is important for the treatment of GVHD [79]. The results are shown in FIG. 14. Mice administered strain NCIMB 43170 maintained greater levels of plasma citrulline in comparison to mice administered butyrate salts (Group 10), which is significant considering the role of butyrate in maintaining correct barrier function. These data demonstrate that compositions comprising bacterial strains of the species Blautia producta and Blautia coccoides may be useful for treating and preventing colitis and other inflammatory and autoimmune diseases.

### Example 2—SCFA Analysis

SCFA from spent media was measured using LC-MS techniques to provide a robust in vitro analysis of the carbohydrate fermentation pathways of bacterial strain NCIMB 43170 and a number of other strains of B. producta/Blautia coccoides—it was not possible to definitively classify the reference strains as belonging to Blautia producta or Blautia coccoides using 16S sequencing and MALDI-TOF MS analysis, due to the unusually high similarity between these two species. SCFA have been implicated in many different diseases and most importantly in inflammation alleviation with the production of butyrate.

### Experimental Design and Methods

Bacterial cultures were inoculated into YCFA and PYG media (10% inoculum), which had been pre-prepared and pre-equilibrated beforehand (at least 24 hours before for pre-equilibration). At 16 hours' post inoculation, 1 mL of the cultures were passed through a 0.22 μM filter and stored in a sterile Eppendorf. The samples were then put into the −80° C. freezer prior to analysis.

Aliquots were stored (n=3) for 2 biological replicates. The analysis was performed on n=2 of the biological replicates, including an internal standard and quality control checks. Optical density readings were recorded for each biological replicate and monitored for differences or deviations from the late log OD limits provided by isolation.

### Results

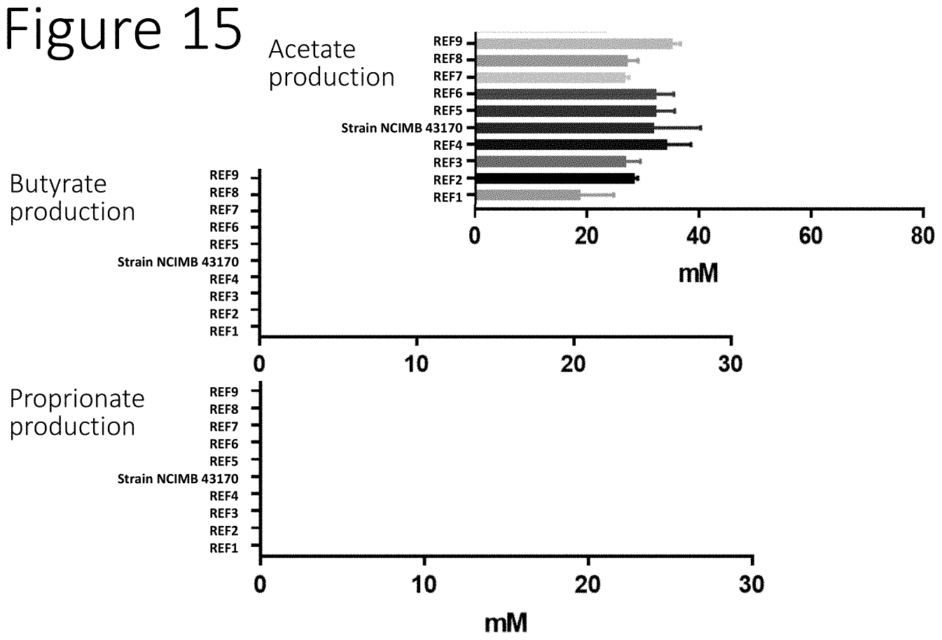

The results are shown in FIG. 15. Bacterial strain NCIMB 43170 and all the reference B. producta/Blautia coccoides strains were found to produce acetate at 20-40 mM. None of the strains tested produced butyrate or propionate. When grown in YCFA, strain NCIMB 43170 produced propanoic acid and smaller quantities of 2-methyl-propanoic acid (isobutyric acid), 3-methyl-butanoic acid (isovaleric acid) and pentanoic acid (data not shown).

### Example 3—Stability Testing

A composition described herein containing at least one bacterial strain described herein is stored in a sealed container at 25° C. or 4° C. and the container is placed in an atmosphere having 30%, 40%, 50%, 60%, 70%, 75%, 80%, 90% or 95% relative humidity. After 1 month, 2 months, 3 months, 6 months, 1 year, 1.5 years, 2 years, 2.5 years or 3 years, at least 50%, 60%, 70%, 80% or 90% of the bacterial strain shall remain as measured in colony forming units determined by standard protocols.

### Example 4—IL-6 Secretion in Human U373 Cells

Blautia coccoides Supernatant Preparation

Two strains of Blautia coccoides (strains A and B) were each cultured separately as follows: 1004, of a Research Cell Bank vial was used to inoculate 10 mL of YCFA+ broth. The culture was incubated overnight in an anaerobic workstation at 37° C. Each overnight culture was used to inoculate five Hungate tubes containing 10 mL of fresh growth medium with a 10% subculture. Culture tubes were incubated until they reached early stationary phase, following which cell-free supernatants (CFS) were collected as follows. Individual culture tubes were combined and the bacterial density (O.D. 600 nm) was recorded. Cell-free supernatants of the two strains were obtained by centrifugation (5000×g for 15 minutes) and filtration through a 0.45 μm followed by a 0.22 μm filter.

### Treatment of U373 Cells

U373 is a human glioblastoma astrocytoma cell line. U373 cells were maintained in 25 ml MEME 4.5 g/L D-glucose supplemented with 10% heat-inactivated FBS, 4 mM L-Glutamine, 100 U/ml penicillin, 100 μg/ml streptomycin and 5 μg/ml plasmocin, 1% Non-Essential Amino Acids, 1% Sodium Pyruvate (full growth media).

Cells were plated in 24-well plates at a density of 100,000 cells/well in 1 ml of full growth media and left to rest at 37° C./5% CO₂ for 72 h. On the day of the treatment, the media was removed from each well, cells were rinsed with 0.5 ml wash media (serum free MEME), 0.9 ml stimulation media (MEME media containing 2% FBS) containing 1 μg/ml LPS was added to the appropriate wells and incubated at 37° C. and 5% CO₂. After 1 h pre-incubation, cells were removed from the CO₂ incubator and treated with 100 μl Blautia coccoides supernatant. YCFA⁺ blank media was used as control. Cells were then incubated for a further 24 h at 37° C./5% CO₂, after which cell-free supernatants were collected and spun down at 10,000 g at 4° C. for 3 minutes. Samples were aliquoted in 1.5 ml microtubes and stored in −80° C. for hIL-6 ELISA.

### Measurement of IL-6 Secretion

IL-6 secretion using Human IL-6 Standard ABTS ELISA Development Kits (Peprotech) were measured in the cell free supernatants from U373 cells. Samples were analysed in accordance to the manufacturer's protocol, absorbance at 405 nm with correction wavelength set at 655 nm was recorded using the iMark microplate reader (Bio-Rad). Raw data were plotted and analysed using GraphPad Prism 7 software.

### Results and Discussion

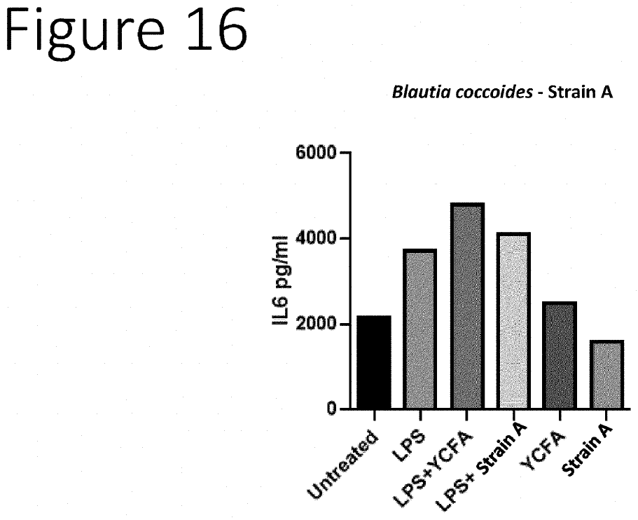

The results are shown in FIGS. 16 and 17. Treatment with each of the Blautia coccoides supernatants led to a reduction in the secretion of IL-6 by U373 cells following immune stimulation relative to cells treated with the YCFA+ medium control.

These data demonstrate that compositions comprising bacterial strains of the species Blautia coccoides are effective in reducing the secretion of the pro-inflammatory cytokine IL-6 and therefore useful for treating and preventing inflammatory and autoimmune diseases.

### Example 5—Occludin Protein Levels

### Treatment of Colorectal Cell Lines

Cells of the colorectal cell line HCT116 were seeded in black 96 well plates at a density of 10,000 cells/well overnight. The cells were treated for 24 hours with 10% bacterial supernatant from Blautia coccoides strain B cultured in YCFA+ media as set out in example 4, with YCFA+ media or with 2 mM butyrate. Indirect immunofluorescence was used to assess the effect of treatment with Blautia coccoides supernatant on the levels of acetylated histone H3 (AcH3), acetylated histone H4 (AcH4) and occludin proteins. Occludin helps to regulate the permeability of the gut epithelium and maintain the gut barrier function. An increase in occludin protein levels is therefore a desirable trait.

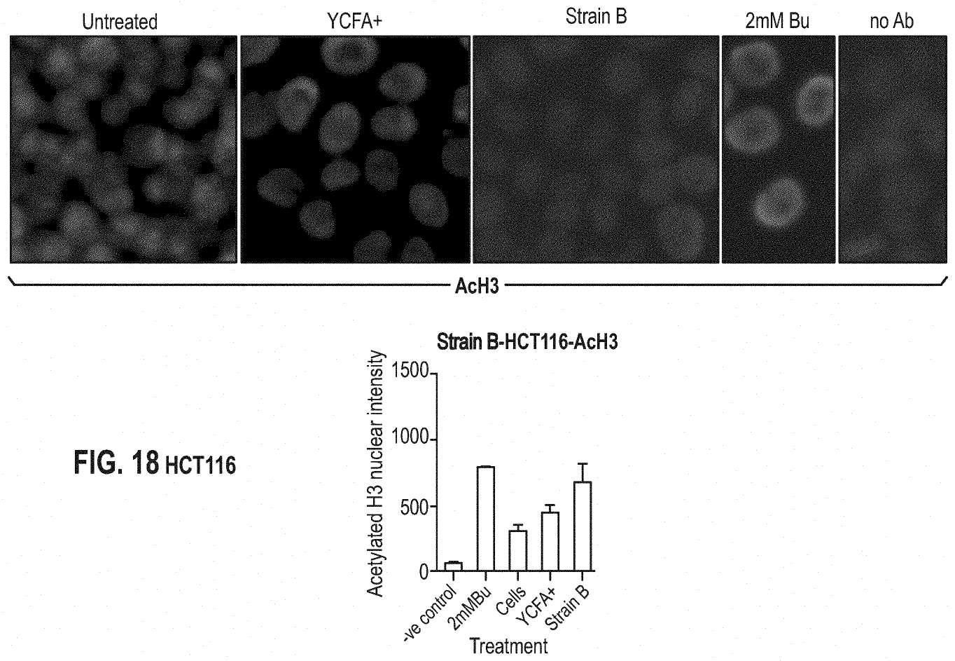

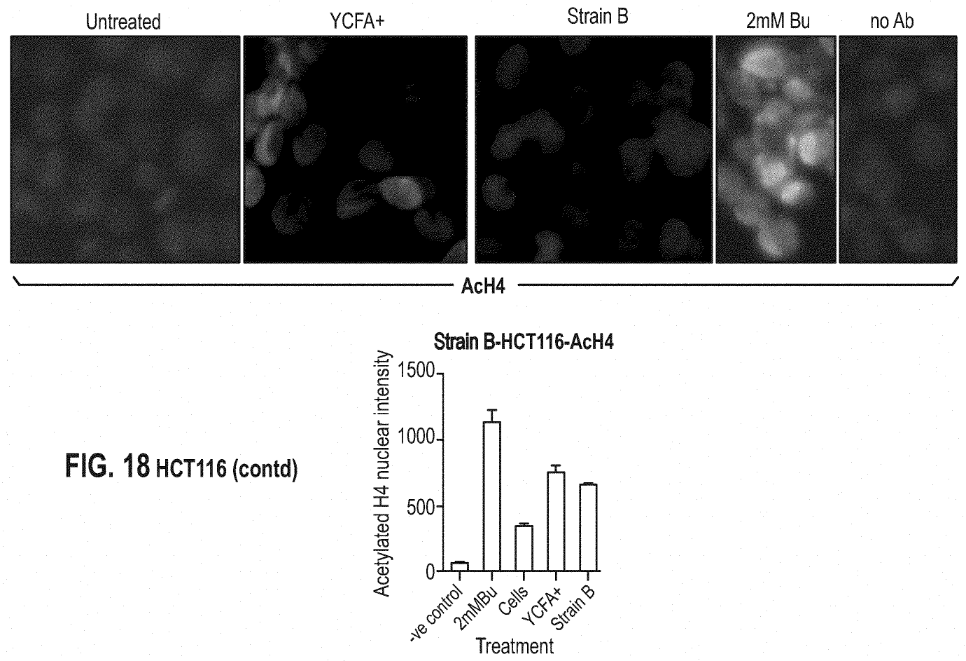

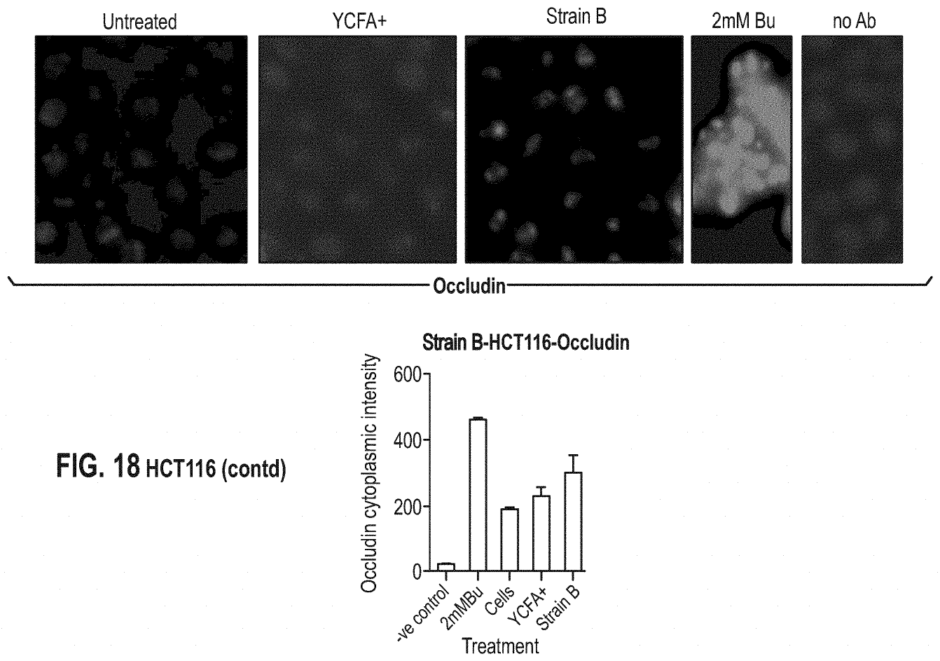

After the treatment, the cells were fixed with 4% paraformaldehyde in PBS (pH 7.3) for 20 minutes at room temperature (RT). Fixed cells were washed with PBS, and permeabilized with 0.5% Triton X-100 in PBS for 10 minutes. After washing with PBS, the plates were incubated with blocking buffer (4% BSA/PBS) for 1 hour at RT before adding the primary antibody for 12 hours at 4° C. (anti-AcH3 antibody (06-599, Millipore) at 1:500, anti-AcH4 (06-598, Millipore) at 1:500), or for 1 hour at 4° C. (anti-Occludin (71-1500; ThermoFisher) at 1:200), diluted in 1% BSA/PBS. The cells were then washed twice with PBS, followed by incubation with Alexa Fluor 488 conjugated anti-rabbit (Molecular Probes Inc) and Alexa Fluor 594 ((Molecular Probes Inc) conjugated for 1 hour at RT. After washing 3 times with PBS, the plates were labelled with DAPI and washed a further with 3 times with PBS. Plates were viewed using ImageExpress PIco microscope (Molecular Devices) equipped with a 20× objective and filter sets suitable for detection of the fluorochromes used. Stored images were saved as TIFF files. Raw analysis data generated by the PICO analysis module were plotted and analysed using GraphPad Prism 7 software Representative images were selected to illustrate the differences in abundance and location of the proteins examined. The results are shown in FIG. 18.

### Results and Discussion

Treatment with Blautia coccoides supernatant resulted in an increase in the levels of AcH3 and occludin relative to untreated cells and YCFA+-treated HCT116 cells (FIG. 18).

These data demonstrate that compositions comprising bacterial strains of the species Blautia coccoides may be effective in maintaining gut barrier function and therefore useful for treating and preventing colitis and other inflammatory and autoimmune diseases.

Sequences
- strain NCIMB 43170 16S rRNA gene sequence -
consensus
SEQ ID NO: 1
GACTTCGGGCGTTACTGACTCCCATGGTGTGACGGGCGGTGTGTACAAGA
CCCGGGAACGTATTCACCGCGGCATTCTGATCCGCGATTACTAGCGATTC
CAGCTTCGTGCAGTCGAGTTGCAGACTGCAGTCCGAACTGGGACGTTATT
TTTGGGATTCGCTCAACATCGCTGTCTCGCTTCCCTTTGTTTACGCCATT
GTAGCACGTGTGTAGCCCAAATCATAAGGGGCATGATGATTTGACGTCGT
CCCCGCCTTCCTCCGGGTTATCCCCGGCAGTCTCCCTAGAGTGCCCACCA
TCATGTGCTGGCTACTAAGGATAAGGGTTGCGCTCGTTGCGGGACTTAAC
CCAACATCTCACGACACGAGCTGACGACAACCATGCACCACCTGTCTCCT
CTGCCCCGAAGGGAAGTCCCCGTTACGGGACGGTCAGAGGGATGTCAAGA
CTTGGTAAGGTTCTTCGCGTTGCTTCGAATTAAACCACATGCTCCACCGC
TTGTGCGGGTCCCCGTCAATTCCTTTGAGTTTCATTCTTGCGAACGTACT
CCCCAGGTGGAATACTTATTGCGTTTGCTGCGGCACCGAATGGCTTTGCC
ACCCGACACCTAGTATTCATCGTTTACGGCGTGGACTACCAGGGTATCTA
ATCCTGTTTGCTCCCCACGCTTTCGAGCCTCAACGTCAGTTACCGTCCAG
TAAGCCGCCTTCGCCACTGGTGTTCCTCCTAATATCTACGCATTTCACCG
CTACACTAGGAATTCCGCTTACCTCTCCGGCACTCTAGAACAACAGTTTC
CAATGCAGTCCTGGGGTTAAGCCCCAGCCTTTCACATCAGACTTGCTCTT
CCGTCTACGCTCCCTTTACACCCAGTAAATCCGGATAACGCTTGCCCCCT
ACGTATTACCGCGGCTGCTGGCACGTAGTTAGCCGGGGCTTCTTAGTCAG
GTACCGTCATTTTCTTCCCTGCTGATAGAAGTTTACATACCGAGATACTT
CTTCCTTCACGCGGCGTCGCTGCATCAGGGTTTCCCCCATTGTGCAATAT
TCCCCACTGCTGCCTCCCGTAGGAGTCTGGGCCGTGTCTCAGTCCCAATG
TGGCCGTTCACCCTCTCAGGCCGGCTACTGATCGTCGCCTTGGTGGGCCG
TTACCCCTCCAACTAGCTAATCAGACGCGGGTCCATCTCATACCACCGGA
GTTTTTCACACCAGACCATGCGGTCCTGTGCGCTTATGCGGTATTAGCAG
TCATTTCTAACTGTTATCCCCCTGTATGAGGCAGGTTACCCACGCGTTAC
TCACCCGTCCGCCGCTCAGTCACAAAGACTTCAATCCGAAGAAATCCTGT
CTTAGTGCTTCGCT
- Blautia producta/Blautia coccoides strain REF1
16S rRNA gene sequence - Contig consensus sequence
2 reads assembled using Geneious
SEQ ID NO: 2
TCGGCAGCTCCTTCCTTTCGGTTAGGTCACTGACTTCGGGCGTTACTGAC
TCCCATGGTGTGACGGGCGGTGTGTACAAGACCCGGGAACGTATTCACCG
CGGCATTCTGATCCGCGATTACTAGCGATTCCAGCTTCGTGCAGTCGAGT
TGCAGACTGCAGTCCGAACTGGGACGTTATTTTTGGGATTCGCTCAACAT
CGCTGTCTCGCTTCCCTTTGTTTACGCCATTGTAGCACGTGTGTAGCCCA
AATCATAAGGGGCATGATGATTTGACGTCGTCCCCGCCTTCCTCCGGGTT
ATCCCCGGCAGTCTCCCTAGAGTGCCCACCATCATGTGCTGGCTACTAAG
GATAAGGGTTGCGCTCGTTGCGGGACTTAACCCAACATCTCACGACACGA
GCTGACGACAACCATGCACCACCTGTCTCCTCTGCCCCGAAGGGAAGTCC
CCGTTACGGGACGGTCAGAGGGATGTCAAGACTTGGTAAGGTTCTTCGCG
TTGCTTCGAATTAAACCACATGCTCCACCGCTTGTGCGGGTCCCCGTCAA
TTCCTTTGAGTTTCATTCTTGCGAACGTACTCCCCAGGTGGAATACTTAT
TGCGTTTGCTGCGGCACCGAATGGCTTTGCCACCCGACACCTAGTATTCA
TCGTTTACGGCGTGGACTACCAGGGTATCTAATCCTGTTTGCTCCCCACG
CTTTCGAGCCTCAACGTCAGTTACCGTCCAGTAAGCCGCCTTCGCCACTG
GTGTTCCTCCTAATATCTACGCATTTCACCGCTACACTAGGAATTCCGCT
TACCTCTCCGGCACTCTAGAACAACAGTTTCCAATGCAGTCCTGGGGTTA
AGCCCCAGCCTTTCACATCAGACTTGCTCTTCCGTCTACGCTCCCTTTAC
ACCCAGTAAATCCGGATAACGCTTGCCCCCTACGTATTACCGCGGCTGCT
GGCACGTAGTTAGCCGGGGCTTCTTAGTCAGGTACCGTCATTTTCTTCCC
TGCTGATAGAAGTTTACATACCGAGATACTTCTTCCTTCACGCGGCGTCG
CTGCATCAGGGTTTCCCCCATTGTGCAATATTCCCCACTGCTGCCTCCCG
TAGGAGTCTGGGCCGTGTCTCAGTCCCAATGTGGCCGTTCACCCTCTCAG
GCCGGCTACTGATCGTCGCCTTGGTGGGCCGTTACCCCTCCAACTAGCTA
ATCAGACGCGGGTCCATCTCATACCACCGGAGTTTTTCACACCAGACCAT
GCGGTCCTGTGCGCTTATGCGGTATTAGCAGTCATTTCTAACTGTTATCC
CCCTGTATGAGGCAGGTTACCCACGCGTTACTCACCCGTCCGCCGCTCAG
TCACAAAGACTTCAATCCGAAGAAATCCGTCTTAGTGCTTCGCTCGACTG
CA
- Blautia producta/Blautia coccoides strain REF2
16S rRNA gene sequence - Contig consensus sequence
2 reads assembled using Geneious
SEQ ID NO: 3
GGTCGCTTCGGCAGCTCTTCCTTTCGGTTAGGTCACTGACTTCGGGCGTT
ACTGACTCCCATGGTGTGACGGGCGGTGTGTACAAGACCCGGGAACGTAT
TCACCGCGGCATTCTGATCCGCGATTACTAGCGATTCCAGCTTCGTGCAG
TCGAGTTGCAGACTGCAGTCCGAACTGGGACGTTATTTTTGGGATTCGCT
CAACATCGCTGTCTCGCTTCCCTTTGTTTACGCCATTGTAGCACGTGTGT
AGCCCAAATCATAAGGGGCATGATGATTTGACGTCGTCCCCGCCTTCCTC
CGGGTTATCCCCGGCAGTCTCCCTAGAGTGCCCACCATCATGTGCTGGCT
ACTAAGGATAAGGGTTGCGCTCGTTGCGGGACTTAACCCAACATCTCACG
ACACGAGCTGACGACAACCATGCACCACCTGTCTCCTCTGCCCCGAAGGG
AAGTCCCCGTTACGGGACGGTCAGAGGGATGTCAAGACTTGGTAAGGTTC
TTCGCGTTGCTTCGAATTAAACCACATGCTCCACCGCTTGTGCGGGTCCC
CGTCAATTCCTTTGAGTTTCATTCTTGCGAACGTACTCCCCAGGTGGAAT
ACTTATTGCGTTTGCTGCGGCACCGAATGGCTTTGCCACCCGACACCTAG
TATTCATCGTTTACGGCGTGGACTACCAGGGTATCTAATCCTGTTTGCTC
CCCACGCTTTCGAGCCTCAACGTCAGTTACCGTCCAGTAAGCCGCCTTCG
CCACTGGTGTTCCTCCTAATATCTACGCATTTCACCGCTACACTAGGAAT
TCCGCTTACCTCTCCGGCACTCTAGAACAACAGTTTCCAATGCAGTCCTG
GGGTTAAGCCCCAGCCTTTCACATCAGACTTGCTCTTCCGTCTACGCTCC
CTTTACACCCAGTAAATCCGGATAACGCTTGCCCCCTACGTATTACCGCG
GCTGCTGGCACGTAGTTAGCCGGGGCTTCTTAGTCAGGTACCGTCATTTT
CTTCCCTGCTGATAGAAGTTTACATACCGAGATACTTCTTCCTTCACGCG
GCGTCGCTGCATCAGGGTTTCCCCCATTGTGCAATATTCCCCACTGCTGC
CTCCCGTAGGAGTCTGGGCCGTGTCTCAGTCCCAATGTGGCCGTTCACCC
TCTCAGGCCGGCTACTGATCGTCGCCTTGGTGGGCCGTTACCCCTCCAAC
TAGCTAATCAGACGCGGGTCCATCTCATACCACCGGAGTTTTTCACACCA
GACCATGCGGTCCTGTGCGCTTATGCGGTATTAGCAGTCATTTCTAACTG
TTATCCCCCTGTATGAGGCAGGTTACCCACGCGTTATCACCCGTCCGCCG
CTCAGTCACAAAGACTTCAATCCGAAGAAATCCGTCTTAGCGCTCCGCTC
GACTGCATGGTAGC

### REFERENCES

[1] Spor et al. (2011) Nat Rev Microbiol. 9(4):279-90.
[2] Eckburg et al. (2005) Science. 10; 308(5728):1635-8.
[3] Macpherson et al. (2001) Microbes Infect. 3(12):1021-35
[4] Macpherson et al. (2002) Cell Mol Life Sci. 59(12):2088-96.
[5] Mazmanian et al. (2005) Cell 15; 122(1):107-18.
[6] Frank et al. (2007) PNAS 104(34):13780-5.
[7] Scanlan et al. (2006) J Clin Microbiol. 44(11):3980-8.
[8] Kang et al. (2010) Inflamm Bowel Dis. 16(12):2034-42.
[9] Machiels et al. (2013) Gut. 63(8):1275-83.
[10] WO 2013/050792
[11] WO 03/046580
[12] WO 2013/008039
[13] WO 2014/167338
[14] Goldin and Gorbach (2008) Clin Infect Dis. 46 Suppl 2:S96-100.
[15] Azad et al. (2013) BMJ. 347:f6471.
[16] WO2017/160944
[17] Liu et al. (2008) Int J Syst Evol Microbiol 58, 1896-1902.
[18] Masco et al. (2003) Systematic and Applied Microbiology, 26:557-563.
[19] Srůtková et al. (2011) J. Microbiol. Methods, 87(1):10-6.
[20] Beres et al., Front Immunol, 2013, 4(163), 1-9.
[21] Graze and Gale, Am J Med, 1979, 66, 611-620.
[22] Yu et al., 2011, Blood, 118(18), 5011-5020
[23] Ye et al. (2015) PLoS One. 10(1):e011770.4
[24] Fabro et al. (2015) Immunobiology. 220(1):124-35
[25] Yin et al. (2014) Immunogenetics. 66(3):215-8
[26] Cheluvappa et al. (2014) Clin Exp Immunol. 175(2):316-22.
[27] Schieck et al. (2014) J Allergy Clin Immunol. 133(3):888-91.
[28] Balato et al. (2014) J Eur Acad Dermatol Venereol. 28(8):1016-24.
[29] Damsker et al., Ann N Y Acad Sci, 2010, 1183, 211-221.
[30] Dardalhon et al., J Autoimmun, 2008, 31(3), 252-256.
[31] Skapenko et al., Arthritis Res Ther, 2005, 7(2), S4-S14.
[32] Mu et al., (2017), Front. Immunol., 8(598), 1-10.
[33] Liu et al., (2005), Acta Paediatr., 94(4), 386-93.
[34] Tlaskalová-Hogenová et al., (2011), Cell. Mol. Immunol., 8(2), 110-120.
[35] Ely, (2018), Clin. Dermatol., 36(3), 376-389.
[36] Fasano & Shea-Donohue, (2005), Nat. Clin. Pract. Gastroenterol. Hepatol., 2(9), 416-422.
[37] Perkley and Maillard (2018) Annual Review of Pathology: Mechanisms of Disease, 13:219-245
[38] Walmsley et al 1998 Gut 43: 29-32
[39] Gasche et al 200 Inflammatory Bowel Diseases 6: 8-15
[40] Zhang et al., World J. Gasfroenfrol., 2014, 20(1), 91-99.
[41] Lacy et al., Gastroenterology, 2016, 150, 1393-1407.
[42] Newcomb & Stokes Peebles, Curr Opin Immunol, 2013, 25(6), 755-760.
[43] Harper & Zeki, Clin Rev Allergy Immunol., 2015, 48(1), 54-65.
[44] Fahy (2009) Proc Am Thorac Soc 6.256-259
[45] Guggino et al., Clin Exp Rheumatol, 2014, 32(1), 77-81.
[46] Li et al., Clin Dev Immunol., 2013, Article ID: 967301.
[47] Miossec and Kolls (2012) Nat Rev Drug Discov. 11(10):763-76.
[48] Yang et al. (2014) Trends Pharmacol Sci. 35(10):493-500.
[49] Shabgah et al. (2014) Postepy. Dermatol. Alergol. 31(4): 256-61
[50] Koenders et al. (2006) J. Immunol. 176:6262-6269.
[51] Chen et at (2011), Clin Dev Immunol., 2012(970789), 1-16
[52] Zhang et al., J Neuroimmunol., 2014, 266(1-2), 56-63
[53] Luan et al., Int Immunopharmacol., 2015, 29(2), 278-284.
[54] Talaat et al., Cytokine, 2015, 72(2), 146-153.
[55] Shah et al., Arthritis Res Ther., 2010, 12(2), R53.
[56] Atalar et al., Curr Opin Organ Transplant, 2009, 14(1) 23-29.
[57] Miyamoto-Shinohara et al. (2008) J. Gen. Appl. Microbiol., 54, 9-24.
[58] Cryopreservation and Freeze-Drying Protocols, ed. by Day and McLellan, Humana Press.
[59] Leslie et al. (1995) Appl. Environ. Microbiol. 61, 3592-3597.
[60] Mitropoulou et al. (2013) J Nutr Metab. (2013) 716861.
[61] Kailasapathy et al. (2002) Curr Issues Intest Microbiol. 3(2):39-48.
[62] Handbook of Pharmaceutical Excipients, 2nd Edition, (1994), Edited by A Wade and PJ Weller
[63] Remington's Pharmaceutical Sciences, Mack Publishing Co. (A. R. Gennaro edit. 1985)
[64] Handbook of Microbiological Media, Fourth Edition (2010) Ronald Atlas, CRC Press.
[65] Maintaining Cultures for Biotechnology and Industry (1996) Jennie C. Hunter-Cevera, Academic Press
[66] Strobel (2009) Methods Mol Biol. 581:247-61.
[67] Gennaro (2000) Remington: The Science and Practice of Pharmacy. 20th edition, ISBN: 0683306472.
[68] Molecular Biology Techniques: An Intensive Laboratory Course, (Ream et al., eds., 1998, Academic Press).
[69] Methods In Enzymology (S. Colowick and N. Kaplan, eds., Academic Press, Inc.)
[70] Handbook of Experimental Immunology, Vols. I-IV (D. M. Weir and C. C. Blackwell, eds, 1986, Blackwell
Scientific Publications)
[71] Sambrook et al. (2001) Molecular Cloning: A Laboratory Manual, 3rd edition (Cold Spring Harbor Laboratory Press).
[72] Handbook of Surface and Colloidal Chemistry (Birdi, K. S. ed., CRC Press, 1997)
[73] Ausubel et al. (eds) (2002) Short protocols in molecular biology, 5th edition (Current Protocols).
[74] PCR (Introduction to Biotechniques Series), 2nd ed. (Newton & Graham eds., 1997, Springer Verlag)
[75] Current Protocols in Molecular Biology (F. M. Ausubel et al., eds., 1987) Supplement 30
[76] Smith & Waterman (1981) Adv. Appl. Math. 2: 482-489
[77] Matthewson et al (2016) Nature Immunology 15: 505-513
[78] Baas, T (2013) SciBX 6: doi:10.1038/scibx.2013.1310
[79] Johansson and Ekman (2004) Blood 104:5075

1.-15. (canceled)

16. A method of treating one or more symptoms of a gastrointestinal inflammatory disease in a subject in need thereof, comprising administering to the subject a therapeutically effective amount of a composition comprising a bacterial strain of the species Blautia producta or Blautia coccoides, wherein the bacterial strain comprises a 16s rRNA gene sequence having at least 95% sequence identity to SEQ ID NO: 1, wherein the one or more symptoms of the gastrointestinal inflammatory disease comprises ulcerations and/or bleeding, weight loss, poor weight gain, or increased gut permeability, and wherein the administering is effective to treat the one or more symptoms of the gastrointestinal inflammatory disease in the subject.

17. The method of claim 16, wherein the gastrointestinal inflammatory disease comprises colitis, Crohn's disease, or inflammatory bowel disease (IBD).

18. The method of claim 17, wherein the colitis comprises ulcerative colitis.

19. The method of claim 18, wherein the ulcerative colitis comprises distal colitis, proctitis, proctosigmoiditis, left-sided colitis, extensive colitis, or pancolitis.

20. The method of claim 17, wherein the Crohn's disease comprises Ileocolic Crohn's, Crohn's ileitis, or Crohn's colitis.

21. The method of claim 16, wherein the administering is effective to reduce ulcerations and/or bleeding, reduce weight loss, enhance weight gain, or reduce gut permeability in the subject with the gastrointestinal inflammatory disease, thereby treating the gastrointestinal inflammatory disease.

22. The method of claim 16, wherein the therapeutically effective amount comprises from about 1×10³ to about 1×10¹¹ colony forming units (CFU).

23. The method of claim 16, wherein the composition comprises the bacterial strain in an amount from about 1×10³ to about 1×10¹¹ colony forming units per gram (CFU/g), with respect to the weight of the composition.

24. The method of claim 16, wherein the bacterial strain is lyophilized.

25. The method of claim 16, wherein the bacterial strain is viable and capable of partially or totally colonizing the intestine.

26. The method of claim 16, wherein the composition comprises no more than de minimis or biologically irrelevant amounts of other bacterial strains or species.

27. The method of claim 16, wherein the composition is encapsulated.

28. The method of claim 16, wherein the administering comprises oral, rectal, subcutaneous, nasal, buccal, or sublingual administration.

29. The method of claim 16, wherein the bacterial strain does not produce butyrate.

30. The method of claim 16, wherein the bacterial strain comprises a 16s rRNA gene sequence that has at least 98% sequence identity to SEQ ID NO: 1.

31. The method of claim 16, wherein the bacterial strain is the strain deposited under accession number NCIMB 43170, or a derivative or a biotype thereof.

32. A pharmaceutical composition comprising a therapeutically effective amount of a bacterial strain of the species Blautia producta and a pharmaceutically acceptable excipient, diluent, or carrier;
wherein the bacterial strain is lyophilized;
wherein the therapeutically effective amount of the bacterial strain comprises from about 1×103 to about 1×1011 colony forming units per gram (CFU/g) with respect to the total weight of the pharmaceutical composition; and
wherein the bacterial strain comprises a 16s rRNA gene sequence of SEQ ID NO: 1

33. A method of treating colitis in a subject with graft versus host disease (GVHD), comprising administering to the subject a therapeutically effective amount of a composition comprising a bacterial strain of the species Blautia producta or Blautia coccoides, wherein the bacterial strain comprises a 16s rRNA gene sequence having at least 95% sequence identity to SEQ ID NO: 1, and wherein the administering is effective to treat colitis in the subject with GVHD.

34. A method of treating one or more symptoms associated with graft versus host disease (GVHD) in a subject in need thereof, comprising administering to the subject a therapeutically effective amount of a composition comprising a bacterial strain of the species Blautia producta or Blautia coccoides, wherein the bacterial strain comprises a 16s rRNA gene sequence having at least 95% sequence identity to SEQ ID NO: 1, wherein the one or more symptoms associated with GVHD comprises weight loss, poor weight gain, poor skin integrity, or increased gut permeability, and wherein the administering is effective to treat the one or more symptoms associated with GVHD in the subject.

35. A method of treating an inflammatory or autoimmune disease in a subject in need thereof, comprising administering to the subject a therapeutically effective amount of a composition comprising a bacterial strain of the species Blautia producta, wherein the bacterial strain comprises a 16s rRNA gene sequence of SEQ ID NO: 1.


SEQUENCE LISTING (3 sequences)

SEQ ID NO: 1 - DNA, 1364 bp, Blautia producta
  Blautia producta
gacttcgggcgttactgactcccatggtgtgacgggcggtgtgtacaagacccgggaacgtattcaccgcggcattctgatccgcgattactagcgattccagcttcgtgcagtcgagttgcagactgcagtccgaactgggacgttatttttgggattcgctcaacatcgctgtctcgcttccctttgtttacgccattgtagcacgtgtgtagcccaaatcataaggggcatgatgatttgacgtcgtccccgccttcctccgggttatccccggcagtctccctagagtgcccaccatcatgtgctggctactaaggataagggttgcgctcgttgcgggacttaacccaacatctcacgacacgagctgacgacaaccatgcaccacctgtctcctctgccccgaagggaagtccccgttacgggacggtcagagggatgtcaagacttggtaaggttcttcgcgttgcttcgaattaaaccacatgctccaccgcttgtgcgggtccccgtcaattcctttgagtttcattcttgcgaacgtactccccaggtggaatacttattgcgtttgctgcggcaccgaatggctttgccacccgacacctagtattcatcgtttacggcgtggactaccagggtatctaatcctgtttgctccccacgctttcgagcctcaacgtcagttaccgtccagtaagccgccttcgccactggtgttcctcctaatatctacgcatttcaccgctacactaggaattccgcttacctctccggcactctagaacaacagtttccaatgcagtcctggggttaagccccagcctttcacatcagacttgctcttccgtctacgctccctttacacccagtaaatccggataacgcttgccccctacgtattaccgcggctgctggcacgtagttagccggggcttcttagtcaggtaccgtcattttcttccctgctgatagaagtttacataccgagatacttcttccttcacgcggcgtcgctgcatcagggtttcccccattgtgcaatattccccactgctgcctcccgtaggagtctgggccgtgtctcagtcccaatgtggccgttcaccctctcaggccggctactgatcgtcgccttggtgggccgttacccctccaactagctaatcagacgcgggtccatctcataccaccggagtttttcacaccagaccatgcggtcctgtgcgcttatgcggtattagcagtcatttctaactgttatccccctgtatgaggcaggttacccacgcgttactcacccgtccgccgctcagtcacaaagacttcaatccgaagaaatcctgtcttagtgcttcgct

SEQ ID NO: 2 - DNA, 1402 bp, Blautia
  Blautia producta or Blautia coccoides
tcggcagctccttcctttcggttaggtcactgacttcgggcgttactgactcccatggtgtgacgggcggtgtgtacaagacccgggaacgtattcaccgcggcattctgatccgcgattactagcgattccagcttcgtgcagtcgagttgcagactgcagtccgaactgggacgttatttttgggattcgctcaacatcgctgtctcgcttccctttgtttacgccattgtagcacgtgtgtagcccaaatcataaggggcatgatgatttgacgtcgtccccgccttcctccgggttatccccggcagtctccctagagtgcccaccatcatgtgctggctactaaggataagggttgcgctcgttgcgggacttaacccaacatctcacgacacgagctgacgacaaccatgcaccacctgtctcctctgccccgaagggaagtccccgttacgggacggtcagagggatgtcaagacttggtaaggttcttcgcgttgcttcgaattaaaccacatgctccaccgcttgtgcgggtccccgtcaattcctttgagtttcattcttgcgaacgtactccccaggtggaatacttattgcgtttgctgcggcaccgaatggctttgccacccgacacctagtattcatcgtttacggcgtggactaccagggtatctaatcctgtttgctccccacgctttcgagcctcaacgtcagttaccgtccagtaagccgccttcgccactggtgttcctcctaatatctacgcatttcaccgctacactaggaattccgcttacctctccggcactctagaacaacagtttccaatgcagtcctggggttaagccccagcctttcacatcagacttgctcttccgtctacgctccctttacacccagtaaatccggataacgcttgccccctacgtattaccgcggctgctggcacgtagttagccggggcttcttagtcaggtaccgtcattttcttccctgctgatagaagtttacataccgagatacttcttccttcacgcggcgtcgctgcatcagggtttcccccattgtgcaatattccccactgctgcctcccgtaggagtctgggccgtgtctcagtcccaatgtggccgttcaccctctcaggccggctactgatcgtcgccttggtgggccgttacccctccaactagctaatcagacgcgggtccatctcataccaccggagtttttcacaccagaccatgcggtcctgtgcgcttatgcggtattagcagtcatttctaactgttatccccctgtatgaggcaggttacccacgcgttactcacccgtccgccgctcagtcacaaagacttcaatccgaagaaatccgtcttagtgcttcgctcgactgca

SEQ ID NO: 3 - DNA, 1414 bp, Blautia
  Blautia producta or Blautia coccoides
ggtcgcttcggcagctcttcctttcggttaggtcactgacttcgggcgttactgactcccatggtgtgacgggcggtgtgtacaagacccgggaacgtattcaccgcggcattctgatccgcgattactagcgattccagcttcgtgcagtcgagttgcagactgcagtccgaactgggacgttatttttgggattcgctcaacatcgctgtctcgcttccctttgtttacgccattgtagcacgtgtgtagcccaaatcataaggggcatgatgatttgacgtcgtccccgccttcctccgggttatccccggcagtctccctagagtgcccaccatcatgtgctggctactaaggataagggttgcgctcgttgcgggacttaacccaacatctcacgacacgagctgacgacaaccatgcaccacctgtctcctctgccccgaagggaagtccccgttacgggacggtcagagggatgtcaagacttggtaaggttcttcgcgttgcttcgaattaaaccacatgctccaccgcttgtgcgggtccccgtcaattcctttgagtttcattcttgcgaacgtactccccaggtggaatacttattgcgtttgctgcggcaccgaatggctttgccacccgacacctagtattcatcgtttacggcgtggactaccagggtatctaatcctgtttgctccccacgctttcgagcctcaacgtcagttaccgtccagtaagccgccttcgccactggtgttcctcctaatatctacgcatttcaccgctacactaggaattccgcttacctctccggcactctagaacaacagtttccaatgcagtcctggggttaagccccagcctttcacatcagacttgctcttccgtctacgctccctttacacccagtaaatccggataacgcttgccccctacgtattaccgcggctgctggcacgtagttagccggggcttcttagtcaggtaccgtcattttcttccctgctgatagaagtttacataccgagatacttcttccttcacgcggcgtcgctgcatcagggtttcccccattgtgcaatattccccactgctgcctcccgtaggagtctgggccgtgtctcagtcccaatgtggccgttcaccctctcaggccggctactgatcgtcgccttggtgggccgttacccctccaactagctaatcagacgcgggtccatctcataccaccggagtttttcacaccagaccatgcggtcctgtgcgcttatgcggtattagcagtcatttctaactgttatccccctgtatgaggcaggttacccacgcgttatcacccgtccgccgctcagtcacaaagacttcaatccgaagaaatccgtcttagcgctccgctcgactgcatggtagc